SEQUENCE LISTING (59 sequences)

SEQ ID NO: 1 - protein, 2366 aa, Clostridium difficile
  MISC_FEATURE: C. difficile toxin B amino acid sequence
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDDKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 2 - protein, 250 aa, Clostridium difficile
  MISC_FEATURE: C. difficile C-terminal CDB-C250 of toxin B amino acid sequence
MQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 3 - protein, 23 aa, Clostridium difficile
  MISC_FEATURE: C. difficile C-terminal repeat A
MQVGFVTINDKVFYFSDSGIIES

SEQ ID NO: 4 - protein, 20 aa, Clostridium difficile
  MISC_FEATURE: C. difficile C-terminal repeat B
GVQNIDDNYFYIDDNGIVQI

SEQ ID NO: 5 - protein, 30 aa, Clostridium difficile
  MISC_FEATURE: C. difficile C-terminal repeat C
GVFDTSDGYKYFAPANTVNDNIYGQAVEYS

SEQ ID NO: 6 - protein, 20 aa, Clostridium difficile
  MISC_FEATURE: C. difficile C-terminal repeat D
GLVRVGEDVYYFGETYTIET

SEQ ID NO: 7 - protein, 24 aa, Clostridium difficile
  MISC_FEATURE: C. difficile C-terminal repeat E
GWIYDMENESDKYYFNPETKKACK

SEQ ID NO: 8 - protein, 20 aa, Clostridium difficile
  MISC_FEATURE: C. difficile C-terminal repeat F
GINLIDDIKYYFDEKGIMRT

SEQ ID NO: 9 - protein, 20 aa, Clostridium difficile
  MISC_FEATURE: C. difficile C-terminal repeat G
GLISFENNNYYFNENGEMQF

SEQ ID NO: 10 - protein, 20 aa, Clostridium difficile
  MISC_FEATURE: C. difficile C-terminal repeat H
GYINIEDKMFYFGEDGVMQI

SEQ ID NO: 11 - protein, 23 aa, Clostridium difficile
  MISC_FEATURE: C. difficile C-terminal repeat I
GVFNTPDGFKYFAHQNTLDENFE

SEQ ID NO: 12 - protein, 27 aa, Clostridium difficile
  MISC_FEATURE: C. difficile C-terminal repeat J
GESINYTGWLDLDEKRYYFTDEYIAAT

SEQ ID NO: 13 - protein, 23 aa, Clostridium difficile
  MISC_FEATURE: C. difficile C-terminal repeat K
GSVIIDGEEYYFDPDTAQLVISE

SEQ ID NO: 14 - protein, 2710 aa, Clostridium difficile
  MISC_FEATURE: C. difficile toxin A amino acid sequence
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNIIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNDSKAVTGWRIINNEKYYFNPNNAIAAYGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 15 - protein, 108 aa, Mus musculus
  MISC_FEATURE: 1C11 IgG1 HC.pro
LEESGAELVKPGASVKLSCTTSGFNIKDTYIHWMKQRPEQGLEWIGRIDPANGNTKYDPKFQDRATITADTSSNTAYLHLSSLTSEDTAVYYCARSTGWYFDVWGAGP

SEQ ID NO: 16 - protein, 101 aa, Mus musculus
  MISC_FEATURE: 3E1 IgG1 HC.pro
EVKLEESGPELVKPGASVKISCKTSGYTFTEYTMHWVKQSHGKSLEWIGGIIPNNGGTSYNQKFKGKATLTVDKSSSTAYMELRSLTSEDSAVYYCARWTT

SEQ ID NO: 17 - protein, 109 aa, Mus musculus
  MISC_FEATURE: 3G8 IgG2b HC.pro
VQLEESGPELVKPGTSVKMSCKASGYTFTSYVMHWVKQKPGQGLEWIGYINPYNDGTKYNEKFKGKATLTSDKSSSTAYMELSSLTSEDSAVYYCTRSAYYRYFDVWGA

SEQ ID NO: 18 - protein, 104 aa, Mus musculus
  MISC_FEATURE: 3H10 IgG1 HC.pro
EESGPELVKPGASMKISCKASGYSFTGYTMNWVKQSHGKNLEWIGLIIPYNGGTSYNQKFKGKATLTVDKSSSTAYMELLSLTSEDSAVYYCARGGLRRAMDYW

SEQ ID NO: 19 - protein, 101 aa, Mus musculus
  MISC_FEATURE: 4B3 IgG1 HC.pro
ESGPDLVAPSQSLSITCTVSGFSLTSYGVHWVRQPPGKGLEWLVVIWTDGSTTYNSALKSRLSISKDNSKSQVFLKMNSLQTDDTAIYYCARQRFYAMDYW

SEQ ID NO: 20 - protein, 88 aa, Mus musculus
  MISC_FEATURE: 1C11 Kappa LC.pro
DIVMTQTPSSLAVSVGEKVTMNCKSSQSLLYSGNQKNYLAWYQQKPGQSPKLLIYWASTRKSGVPDRFTGSGSGTDFTLTISSVKAED

SEQ ID NO: 21 - protein, 89 aa, Mus musculus
  MISC_FEATURE: 3E1 Kappa LC.pro
IMSASPGEKVTITCSARSSVSYMHWFQQKPGTSPKLWIYSTSNLASGVPARFSGSGSGTSYSLTISRMEAEDAATYYCQQRSSYPFTFG

SEQ ID NO: 22 - protein, 91 aa, Mus musculus
  MISC_FEATURE: 3G8 Kappa LC.pro
SSLSASLGERVSLTCRASQDIGSSLNWLQQEPDGTIKRLIYATSSLDSGVPKRFSGSRSGSDYSLTISSLESEDFVDYYCLQYASSPYTFG

SEQ ID NO: 23 - protein, 96 aa, Mus musculus
  MISC_FEATURE: 3H10 Kappa LC.pro
HQSPSSLSASLGERVSLTCRASQDIGSSLNWLQQEPDGTIKRLIYATSSLDSGVPKRFSGSRSGSDYSLTISSLESEDFVDYYCLQYASSPWTFGG

SEQ ID NO: 24 - protein, 101 aa, Mus musculus
  MISC_FEATURE: 4B3 Kappa LC.pro
SPLSLPVSLGDQASISCRSSQSLVHSNGNTHLHWYLQKPGQSPKLLIYKVSNRFSGVPDRFSGSGSGTEFTLKISRVEAEDLGVYFCSQSTHVPLTFGAGT

SEQ ID NO: 25 - protein, 17 aa, Mus musculus
  MISC_FEATURE: 1C11 Kappa LC.pro CDR1
KSSQSLLYSGNQKNYLA

SEQ ID NO: 26 - protein, 7 aa, Mus musculus
  MISC_FEATURE: 1C11 Kappa LC.pro CDR2
IYWASTR

SEQ ID NO: 27 - protein, 10 aa, Mus musculus
  MISC_FEATURE: 3E1 Kappa LC.pro CDR1
SARSSVSYMH

SEQ ID NO: 28 - protein, 7 aa, Mus musculus
  MISC_FEATURE: 3E1 Kappa LC.pro CDR2
IYSTSNL

SEQ ID NO: 29 - protein, 9 aa, Mus musculus
  MISC_FEATURE: 3E1 Kappa LC.pro CDR3
QQRSSYPFT

SEQ ID NO: 30 - protein, 11 aa, Mus musculus
  MISC_FEATURE: 3G8 Kappa LC.pro CDR1
RASQDIGSSLN

SEQ ID NO: 31 - protein, 7 aa, Mus musculus
  MISC_FEATURE: 3G8 Kappa LC.pro CDR2
IYATSSL

SEQ ID NO: 32 - protein, 9 aa, Mus musculus
  MISC_FEATURE: 3G8 Kappa LC.pro CDR3
LQYASSPYT

SEQ ID NO: 33 - protein, 11 aa, Mus musculus
  MISC_FEATURE: 3H10 Kappa LC.pro CDR1
RASQDIGSSLN

SEQ ID NO: 34 - protein, 7 aa, Mus musculus
  MISC_FEATURE: 3H10 Kappa LC.pro CDR2
IYATSSL

SEQ ID NO: 35 - protein, 9 aa, Mus musculus
  MISC_FEATURE: 3H10 Kappa LC.pro CDR3
LQYASSPWT

SEQ ID NO: 36 - protein, 16 aa, Mus musculus
  MISC_FEATURE: 4B3 Kappa LC.pro CDR1
RSSQSLVHSNGNTHLH

SEQ ID NO: 37 - protein, 7 aa, Mus musculus
  MISC_FEATURE: 4B3 Kappa LC.pro CDR2
IYKVSNR

SEQ ID NO: 38 - protein, 9 aa, Mus musculus
  MISC_FEATURE: 4B3 Kappa LC.pro CDR3
SQSTHVPLT

SEQ ID NO: 39 - protein, 10 aa, Mus musculus
  MISC_FEATURE: 1C11 IgG1 HC pro CDR1
GFNIKDTYIH

SEQ ID NO: 40 - protein, 18 aa, Mus musculus
  MISC_FEATURE: 1C11 IgG1 HC pro CDR2
RIDPANGNTKYDPKFQDR

SEQ ID NO: 41 - protein, 8 aa, Mus musculus
  MISC_FEATURE: 1C11 IgG1 HC pro CDR3
STGWYFDV

SEQ ID NO: 42 - protein, 10 aa, Mus musculus
  MISC_FEATURE: 3E1 IgG1 HC pro CDR1
GYTFTEYTMH

SEQ ID NO: 43 - protein, 18 aa, Mus musculus
  MISC_FEATURE: 3E1 IgG1 HC pro CDR2
GIIPNNGGTSYNQKFKGK

SEQ ID NO: 44 - protein, 3 aa, Mus musculus
  MISC_FEATURE: 3E1 IgG1 HC pro CDR3
WTT

SEQ ID NO: 45 - protein, 10 aa, Mus musculus
  MISC_FEATURE: 3G8 IgG2b HC pro CDR1
GYTFTSYVMH

SEQ ID NO: 46 - protein, 18 aa, Mus musculus
  MISC_FEATURE: 3G8 IgG2b HC pro CDR2
YINPYNDGTKYNEKFKGK

SEQ ID NO: 47 - protein, 9 aa, Mus musculus
  MISC_FEATURE: 3G8 IgG2b HC pro CDR3
SAYYRYFDV

SEQ ID NO: 48 - protein, 10 aa, Mus musculus
  MISC_FEATURE: 3H10 IgG1 HC pro CDR1
GYSFTGYTMN

SEQ ID NO: 49 - protein, 18 aa, Mus musculus
  MISC_FEATURE: 3H10 IgG1 HC pro CDR2
LIIPYNGGTSYNQKFKGK

SEQ ID NO: 50 - protein, 9 aa, Mus musculus
  MISC_FEATURE: 3H10 IgG1 HC pro CDR3
GGLRRAMDY

SEQ ID NO: 51 - protein, 10 aa, Mus musculus
  MISC_FEATURE: 4B3 IgG1 HC pro CDR1
GFSLTSYGVH

SEQ ID NO: 52 - protein, 17 aa, Mus musculus
  MISC_FEATURE: 4B3 IgG1 HC pro CDR2
VIWTDGSTTYNSALKSR

SEQ ID NO: 53 - protein, 8 aa, Mus musculus
  MISC_FEATURE: 4B3 IgG1 HC pro CDR3
QRFYAMDY

SEQ ID NO: 54 - protein, 11 aa, Mus musculus
  misc_feature (6)..(6): Xaa can be any naturally occurring amino acid
RASQSXGSSLX

SEQ ID NO: 55 - protein, 7 aa, Mus musculus
  misc_feature (6)..(6): Xaa can be any naturally occurring amino acid
IYATSXL

SEQ ID NO: 56 - protein, 9 aa, Mus musculus
  misc_feature (8)..(8): Xaa can be any naturally occurring amino acid
LQYASSPXT

SEQ ID NO: 57 - protein, 10 aa, Mus musculus
  misc_feature (3)..(3): Xaa can be any naturally occurring amino acid
GYXFTSYTMH

SEQ ID NO: 58 - protein, 18 aa, Mus musculus
  misc_feature (1)..(1): Xaa can be any naturally occurring amino acid
XIIPYNGGTXYNQKFKGK

SEQ ID NO: 59 - protein, 8 aa, Mus musculus
  misc_feature (3)..(3): Xaa can be any naturally occurring amino acid
STXRXXDX

(19) United States
(12) Patent Application Publication
(10) Pub. No.: US 2012/0282274 A1
(43) Pub. Date: Nov. 8, 2012

(54) TARGETING OF THE C-TERMINAL SEGMENT OF C.DIFFICILE TOXIN B FOR IMPROVED CLINICAL DIAGNOSIS, PREVENTION, AND TREATMENT

(75) Inventors: Jian-Ping Jin, Northbrook, IL (US); Lance R. Peterson, Winnetka, IL (US)

(73) Assignee: NORTHSHORE UNIVERSITY HEALTH SYSTEM RESEARCH INSTITUTE, Evanston, IL (US)

(21) Appl. No.: 13/508,791

(22) PCT Filed: Nov. 22, 2010
(86) PCT No.: PCT/US10/57660

§ 371 Date: Jul. 10, 2012

Related U.S. Application Data
(60) Provisional application No. 61/263,199, filed on Nov. 20, 2009.

(51) Int. Cl. C07K 16/12; C12N 5/16; C07K 14/33; C12N 15/31; C12N 15/63; C12Q 1/68; A61K 39/08; A61K 39/40; C12N 1/21; G01N 33/577; G01N 33/566; G01N 33/569; A61P 37/04; A61P 31/04; C07K 19/00

(52) U.S. Cl. 424/167.1; 530/389.5; 530/388.4; 530/387.9; 530/387.3; 435/340; 530/350; 530/326; 530/324; 530/325; 536/23.7; 435/320.1; 435/6.12; 424/190.1; 435/252.33; 436/501; 435/7.92; 435/7.1

(57) ABSTRACT

The disclosure provides specific and sensitive anti-toxin B antibodies and fragments thereof suitable for diagnosing Clostridium difficile infection. The antibodies and fragments recognize an epitope in the C-terminal 250-amino-acid region of toxin B of C. difficile, including epitopes defined by protein repeat sequences in this region of toxin B. This disclosure also provides the toxin B-specific epitope in the C-terminal 250-amino-acid region of toxin B of C. difficile for use in vaccine development as well as in the treatment of CDI disease and in treatment of the relapse of CDI disease. Also provided are toxin B polypeptides lacking the cytotoxic domain useful in treating or preventing CDI disease. PCR-based diagnostic assays targeting the 750-nucleotide region at the 3′ end of tcdB are also provided.

### FIELD

The disclosure generally relates to the field of infectious diseases and, more particularly, to the diseases collectively referred to as Clostridium difficile infection (CDI).

### BACKGROUND

Clostridium difficile-Associated Disease (CDAD), now renamed as C. difficile infection (CDI), is a significant health concern worldwide, resulting from the colonization and subsequent infection of the bowel (colon) by C. difficile, a Gram-positive, spore-forming, obligate anaerobic bacterium. CDI is a worldwide problem, typically of nosocomial origin, and is presently diagnosed at a rate approximating 1% of hospital admissions at a conservative estimated treatment cost of $10,000.00 per patient. CDI is a disease causing diarrhea and/or colitis (including pseudomembranous colitis), and C. difficile has been associated with inflammatory bowel disease. In severe cases, CDI can be fatal and, in economic terms, CDI is a significant health problem, identified as the leading cause of nosocomial diagnosed infectious diarrhea, affecting an estimated three million hospitalized patients per year in the U.S. alone.

Treatment of patients with antibiotics such as ampicillin, amoxicillin, cephalosporin, and clindamycin disturbs the normal intestinal flora, providing an opportunity for C. difficile to colonize the colon and cause CDI. The onset of CDI typically occurs four to nine days after antibiotic treatment begins, but can also occur for as long as two months after discontinuation of antibiotic therapy. C. difficile can produce symptoms ranging from mild to severe diarrhea and colitis, including pseudomembranous colitis (PMC), a severe form of colitis characterized by abdominal pain, watery diarrhea, and systemic illness (e.g., fever, nausea). Relapse occurs in about 20% of patients following initial treatment; relapsing patients also have increased risk of additional relapses.

C. difficile produces two exotoxins, i.e., toxin A, or enterotoxin, and toxin B, or cytotoxin. Toxin A is reportedly responsible for the increase in intestinal permeability associated with disease. Toxin B, however, is significantly more cytotoxic than toxin A upon exposure to cells in vitro. CDI has been thought to be caused by the actions of these two exotoxins on colonic epithelium. Both toxins are high molecular weight proteins (280-300 kDa) that catalyze covalent modification of Rho proteins, ultimately leading to the depolymerization of actin filaments and cell death.

In the past, CDI has been diagnosed using cytotoxicity assays and immunoassays. Cytotoxicity assays, however, are labor intensive and can require days before results are known. Enzyme immunoassays of stool filtrate offered the promise of a rapid and relatively inexpensive diagnostic assay, but a number of attempts to develop reliable enzyme immunoassays have yielded equivocal results. Frequently, cross reactivity of antibodies elicited with toxin A will cross react with toxin B or with unknown small molecular weight proteins. The resulting diagnoses are compromised by uncertainty as to the source of any immunoassay signal. More particularly, current clinical laboratory diagnostic tests for CDI are enzyme immunoassays (EIAs) that only detect toxin A or that detect both toxin A and toxin B. Currently available EIAs designed to detect only toxin B have failed due to poor sensitivity, due in large measure to the poor immunogenicity of toxin B.

In particular, U.S. Patent Publication No. 20050287150 purports to disclose anti-toxin B antibodies, but the reference taught the denaturation of toxin B with UDP-dialdehyde and use of the resulting toxoid B as an immunogen. Moreover, each such antibody disclosed in that publication exhibited detectable cross-reactivity to toxin A, diminishing the value of such antibodies in methods for detecting C. difficile toxin B or in methods of diagnosing CDI based on the presence of toxin B. One such antibody recognized an epitope mapping to the C-terminal 589 amino acids, establishing that this region of toxin B was insufficiently distinct from toxin A to reduce cross-reactivity to undetectable levels. Consistently, the publication disclosed the use of the cross-reacting anti-toxin B antibodies as a supplement to the use of anti-toxin A antibodies that rendered the anti-toxin A antibodies fully protective against C. difficile challenge in vivo. Further, U.S. Pat. No. 5,231,003 disclosed the generation of anti-toxin B antibodies using toxoid B, i.e., toxin B inactivated with SDS or formaldehyde. These antibodies exhibited sensitivities in the form of detection thresholds too high to be of use, likely due to the use of denatured holo-toxin B (denatured intact toxin B) as the immunogen. Accordingly, there remains a need in the art for rapid and reliable toxin B-specific diagnostics of CDI.

Current CDI treatments are also imperfect, in part because antibiotics remain the treatment of choice notwithstanding the fact that antibiotics also trigger CDI episodes. Antibiotics least likely to cause CDI, such as vancomycin, are frequently used. Vancomycin resistance in other microorganisms, particularly in opportunistic human pathogens, is a cause for concern in using this antibiotic for treatment. Probiotic approaches designed to re-populate the intestinal flora upon antibiotic administration are also known but have not come into widespread use due to low treatment success and because some probiotic microbes have actually caused bacteremia upon administration.

The prevention of CDI, for example by vaccination, has also received some attention. Vaccines have been developed that protect animals from lethal challenge in infectious models of disease. In addition, polyclonal antibodies have been reported to exhibit a protective effect in an animal model of CDI, for example by binding to C. difficile toxins A and B. Further, monoclonal antibodies have also reportedly been isolated that bind to C. difficile toxins and neutralize their activities in vivo and in vitro. There are also reports that human polyclonal antibodies containing toxin-neutralizing antibodies can prevent C. difficile relapse. Further, while it has been reported and generally accepted that antibody response to toxin A is correlated with disease outcome, indicating the efficacy of humoral responses to toxin A in controlling infection, treatment with a toxin A monoclonal antibody failed and the clinical trial was stopped.

Despite the earlier emphasis on the central role of C. difficile toxin A in CDI, there have been no significant improvements in the accuracy and reliability of diagnostics and progress in preventing and/or treating CDI remains an elusive goal (Peterson et al., Ann Intern Med. 151:176-179 (2009)). Accordingly, a need remains in the art for compositions and methods that provide accurate, reliable, quick and cost-effective diagnostics for C. difficile Infection, as well as compositions and methods for the prevention and/or treatment of CDI.

### SUMMARY

The disclosure satisfies at least one of the aforementioned needs in the art by providing reagents that specifically bind to the C-terminal region of Clostridium difficile toxin B or that compete with toxin B. More particularly, the disclosure provides antibodies and antibody fragments, in any known form known in the art, that specifically bind to toxin B and that do not detectably bind, or cross-react, with toxin A of C. difficile. The anti-toxin B antibodies, moreover, have a lower limit of detection (LOD) or greater sensitivity (i.e., more sensitive) than is known in the art. Further, the anti-toxin B antibodies according to the disclosure exhibit binding affinities for a toxin B product of at least 10⁸ M⁻¹, 10⁹ M⁻¹, 10¹⁰ M⁻¹, 10¹¹ M⁻¹, 10¹² M⁻¹ or 10¹³ M⁻¹, wherein the affinity of binding to a toxin B product is at least 10-fold greater than the binding affinity for a non-specific protein. The reagents are useful in methods of detecting the presence of C. difficile toxin B in a biological sample or a fluid exposed to a biological sample and in methods of detecting the presence of C. difficile, e.g., pathogenic C. difficile, by assaying for the specific presence of the virulence factor for C. difficile, toxin B. The reagents are further useful in methods of diagnosing the presence of pathogenic C. difficile and in methods of diagnosing Clostridium difficile infection, or CDI, e.g., C. difficile-associated diarrhea.

The disclosure also provides polypeptides that are fragments of toxin B that compete with intact toxin B under physiological conditions. The polypeptides are derived from the C-terminal 250-amino-acid region of toxin B. A polypeptide containing all of the 250 amino acids derived from the C-terminus of toxin B is designated CDB-C250. The CDB-C250 polypeptide, as well as fragments of CDB-C250, lack the cytotoxic domain of intact toxin B. The CDB-C250 fragments contain at least one of the repeat elements identified in FIG. 2. Each of these repeat elements is about 20 amino acids in length. These polypeptides are able to competitively interfere with toxin B and are, thus, prophylactically and therapeutically useful in preventing or treating CDI.

In one aspect, the disclosure provides an antibody or antibody fragment that specifically binds to the C-terminal 250-amino-acid region of Clostridium difficile toxin B and that does not detectably bind to C. difficile toxin A. The antibody or antibody fragment can be any form of antibody known in the art, such as a full-length polyclonal antibody, a full-length monoclonal antibody, a bioengineered V region polypeptide, or fragments of these antibody forms. An antibody according to the disclosure can be derived from any class, such as an immunoglobulin G, A, or M or IgG, IgA, or IgM antibody, and can be of any sub-class, such as an IgG1, IgG2, IgG3, or IgG4 antibody. The antibody can be a humanized or human antibody, a chimeric antibody, or a CDR-grafted antibody. Moreover, an antibody fragment according to the disclosure comprises the antigen binding site of the parent antibody and includes, e.g., a Fab fragment, a Fab′ fragment, a F(ab′)2 fragment, an Fv fragment, a single-chain antibody, a single-chain Fᵥ (i.e., scFv) molecule, a linear antibody, a diabody, a peptibody, a bi-body (bispecific Fab-scFv), a tribody (Fab-(scFv)2), a hinged or hingeless minibody, a mono- or bi-specific antibody, and antibody fusion proteins comprising the antigen binding site of the parent antibody.

In some embodiments, the antibody or antibody fragment specifically binds to a polypeptide comprising the sequence of a repeat element found in the C-terminal 250 amino acids of toxin B, such as the sequence set forth in any of SEQ ID NOS:3-13. In another embodiment, the antibody or antibody fragment is produced by a hybridoma selected from the group consisting of 1C11, 2C10, 3E1, 3G8, 3H10 and 4B3. For ease of exposition, recitations of “antibody” throughout this document refer to an antibody or to an antibody and a fragment of that antibody, discernable from context. Typically, and therefore when there is any doubt, “antibody” refers to antibody and a fragment thereof. In one embodiment, the hybridoma is the 3H10 hybridoma. Other antibodies and antibody fragments according to the disclosure specifically bind to the epitope specifically bound by an antibody or antibody fragment described above, e.g., an antibody that specifically binds to an epitope in the C-terminal 250-amino-acid region of C. difficile toxin B, such as a polypeptide comprising the sequence set forth in any of SEQ ID NOS:2-13. Additionally, the antibody or antibody fragment as described above may further comprise a second polypeptide covalently bound to the antibody or antibody fragment or constructed/expressed in a fusion polypeptide, for example an antibody or antibody fragment described above wherein the second polypeptide is a cytotoxic polypeptide. The antibody or antibody fragment may also be associated with a non-proteinaceous cytotoxin. In some embodiments, the antibody or antibody fragment is labeled.

In accordance with this aspect of the disclosure, some embodiments provide an antibody that binds to the C-terminal 250-amino-acid region of Clostridium difficile toxin B with an affinity of at least 10⁸ M⁻¹ and comprises: (a) a heavy chain CDR1 amino acid sequence selected from the group consisting of SEQ ID NOS: 39, 42, 45, 48, 51 and a variant thereof in which at most two amino acids have been changed, or a consensus sequence thereof (SEQ ID NO:57); (b) a heavy chain CDR2 amino acid sequence selected from the group consisting of SEQ ID NOS: 40, 43, 46, 49, 52 and a variant thereof in which at most two amino acids have been changed or a consensus sequence thereof (SEQ ID NO:58); and (c) a heavy chain CDR3 amino acid sequence selected from the group consisting of SEQ ID NOS: 41, 44, 47, 50, 53 and a variant thereof in which at most two amino acids have been changed, or a consensus sequence thereof (SEQ ID NO:59). In some embodiments, the antibody comprises a consensus sequence set forth in FIG. 4 and in SEQ ID NOS:57-59 for one or more of the heavy chain CDR1, CDR2 or CDR3 amino acid sequences.

Some of the antibodies according to this aspect of the disclosure comprise a heavy-chain CDR1 sequence and a heavy-chain CDR2 sequence of a hybridoma antibody disclosed herein or a consensus sequence, or a heavy-chain CDR2 sequence and a heavy-chain CDR3 sequence from such a hybridoma or a consensus sequence, or a heavy-chain CDR1 sequence and a heavy-chain CDR3 sequence from such a hybridoma or a consensus sequence. In particular, an antibody comprises SEQ ID NOS: 39 and 40, 42 and 43, 45 and 46, 48 and 49, 51 and 52, or 57 and 58 for the heavy-chain CDR1 and CDR2 sequences of hybridoma 1C11, 3E1, 3G8, 3H10, 4B3, or consensus sequences, respectively. Also, an antibody comprises SEQ ID NOS: 40 and 41, 43 and 44, 46 and 47, 49 and 50, 52 and 53, or 58 and 59 for the heavy-chain CDR2 and CDR3 sequences of hybridoma 1C11, 3E1, 3G8, 3H10, 4B3, or consensus sequences, respectively. Further contemplated is an antibody that comprises SEQ ID NOS: 39 and 41, 42 and 44, 45 and 47, 48 and 50, 51 and 53, or 57 and 59 for the heavy-chain CDR1 and CDR3 sequences of hybridoma 1C11, 3E1, 3G8, 3H10, 4B3, or consensus sequences, respectively. Additionally, an antibody wherein one or more of the heavy chain CDR1, CDR2 or CDR3 amino acid sequences is a consensus sequence as set forth in FIG. 4 and in SEQ ID NOS:57-59 is contemplated.

In some embodiments according to this aspect the antibody comprises (a) an amino acid in a heavy chain CDR1 amino acid sequence that is replaced with an amino acid from a corresponding position within a different heavy chain CDR1 amino acid sequence set forth in FIG. 4 and the sequence listing; (b) an amino acid in a heavy chain CDR2 amino acid sequence that is replaced with an amino acid from a corresponding position within a different heavy chain CDR2 amino acid sequence set forth in FIG. 4 and the sequence listing; or (c) an amino acid in a heavy chain CDR3 amino acid sequence that is replaced with an amino acid from a corresponding position within a different heavy chain CDR3 amino acid sequence set forth in FIG. 4 and the sequence listing. In some embodiments, the antibody or antibody fragment comprises an amino acid sequence that is at least 95% identical to a heavy chain variable region amino acid sequence set forth in FIG. 4 and the sequence listing, including but not limited to, an antibody that comprises a heavy chain variable region amino acid sequence set forth in FIG. 4 and the sequence listing. The antibody according to this aspect is, in some embodiments, an antibody in which one or more heavy chain framework amino acids have been replaced with corresponding amino acid(s) from another human antibody heavy chain framework amino acid sequence.

Some embodiments of this aspect involve an antibody that binds the C-terminal 250-amino-acid region of Clostridium difficile toxin B with an affinity of at least 10⁸ M⁻¹ that comprises: (a) a light chain CDR1 amino acid sequence selected from the group consisting of SEQ ID NOS: 25, 27, 30, 33, 36 and a variant thereof in which at most two amino acids have been changed, or a consensus sequence thereof (SEQ ID NO:54); (b) a light chain CDR2 amino acid sequence selected from the group consisting of SEQ ID NOS: 26, 28, 31, 34, 37 and a variant thereof in which at most two amino acids have been changed, or a consensus sequence thereof (SEQ ID NO:55); and (c) a light chain CDR3 amino acid sequence selected from the group consisting of SEQ ID NOS: 29, 32, 35, 38 and a variant thereof in which at most two amino acids have been changed, or a consensus sequence thereof (SEQ ID NO:56). Embodiments according to this aspect are drawn to an antibody as described herein, wherein one or more of the light chain CDR1, CDR2 or CDR3 amino acid sequences is a consensus sequence set forth in FIG. 4 and in SEQ ID NOS:54-56.

Some of the antibodies or antibody fragments according to this aspect of the disclosure comprise a light-chain CDR1 sequence and a light-chain CDR2 sequence of a hybridoma antibody disclosed herein, or a light-chain CDR2 sequence and a light-chain CDR3 sequence from such a hybridoma, or a light-chain CDR1 sequence and a light-chain CDR3 sequence from such a hybridoma. In particular, an antibody or antibody fragment comprises SEQ ID NOS: 25 and 26, 27 and 28, 30 and 31, 33 and 34, 36 and 37, or 54 and 55, for the light-chain CDR1 and CDR2 sequences of hybridoma 1C11, 3E1, 3G8, 3H10, 4B3, or consensus sequences, respectively. Also, an antibody or antibody fragment comprises SEQ ID NOS: 28 and 29, 31 and 32, 34 and 35, 37 and 38, or 55 and 56, for the light-chain CDR2 and CDR3 sequences of hybridoma 3E1, 3G8, 3H10, 4B3, or consensus sequences, respectively. Further contemplated is an antibody or antibody fragment that comprises SEQ ID NOS: 27 and 29, 30 and 32, 33 and 35, 36 and 38, or 55 and 56, for the light-chain CDR1 and CDR3 sequences of hybridoma 3E1, 3G8, 3H10, 4B3, or consensus sequences, respectively.

In some embodiments, the antibody or antibody fragment described herein is an antibody or fragment thereof wherein (a) an amino acid in a light chain CDR1 amino acid sequence is replaced with an amino acid from a corresponding position within a different light chain CDR1 amino acid sequence set forth in FIG. 4 and the sequence listing; (b) an amino acid in a light chain CDR2 amino acid sequence is replaced with an amino acid from a corresponding position within a different light chain CDR2 amino acid sequence set forth in FIG. 4 and the sequence listing; or (c) an amino acid in a light chain CDR3 amino acid sequence is replaced with an amino acid from a corresponding position within a different light chain CDR3 amino acid sequence set forth in FIG. 4 and the sequence listing. In some embodiments, the antibody or antibody fragment comprises an amino acid sequence that is at least 95% identical to a light chain variable region amino acid sequence set forth in FIG. 4 and the sequence listing, including but not limited to, an antibody or fragment thereof that comprises a light chain variable region amino acid sequence set forth in FIG. 4 and the sequence listing. The antibody or antibody fragment according to this aspect is, in some embodiments, an antibody or fragment thereof in which one or more light chain framework amino acids have been replaced with corresponding amino acid(s) from another human antibody light chain framework amino acid sequence.

In a related aspect, the disclosure provides a hybridoma producing the antibody or antibody fragment described above. In an embodiment, the hybridoma is selected from the group consisting of 1C11, 2C10, 3E1, 3G8, 3H10 and 4B3. In one embodiment, the hybridoma is the 3H10 hybridoma. In some embodiments, the hybridoma produces any of the antibodies or antibody fragments described herein that specifically bind to C. difficile toxin B, e.g., the C-terminal CDB-C250 toxin B peptide fragment or a fragment comprising a repeat motif from the C-terminal 250-amino-acid domain described herein (SEQ ID NOS:3-13) and illustrated in FIG. 2.

Another aspect is a polypeptide comprising a fragment of Clostridium difficile toxin B, wherein the fragment consists of a sequence selected from the group consisting of SEQ ID NOS:2-13. In some embodiments, the polypeptide fragment is the C-terminal 250-amino-acid fragment of Clostridium difficile toxin B comprising the sequence set forth as SEQ ID NO:2. In a related aspect, the disclosure provides a vaccine polypeptide comprising a fragment of Clostridium difficile toxin B, wherein the sequence of the polypeptide is selected from the group consisting of SEQ ID NOS:2-13. An embodiment of this aspect of the disclosure is a vaccine polypeptide wherein the sequence of the polypeptide is set forth in SEQ ID NO:2.

In another aspect, the disclosure provides a polynucleotide comprising the polynucleotide sequence encoding an amino acid sequence selected from the group consisting of SEQ ID NOS:2-13. The disclosure also provides polynucleotides that hybridize under stringent hybridization conditions, or that are at least 95%, 99% or 99.5% identical in sequence to a polynucleotide encoding an amino acid sequence set forth in any of SEQ ID NOS:2-13. Further comprehended is a polynucleotide encoding an antibody or antibody fragment, or domain of such an antibody or antibody fragment, according to the disclosure. A related aspect of the disclosure is a vector comprising any one or more of the polynucleotides described above. Another related aspect is a host cell comprising the vector described above. Any empty vector known in the art is contemplated for placement of a polynucleotide according to the disclosure, and any host cell known in the art is contemplated for placement of the vector according to the disclosure.

Another aspect of the disclosure is a method for detecting the presence of Clostridium difficile toxin B in a sample comprising: (a) contacting the sample with an antibody or antibody fragment as described above under conditions suitable for binding; and (b) detecting the binding of the antibody or antibody fragment to Clostridium difficile toxin B, wherein the binding of the antibody or antibody fragment to toxin B is diagnostic of Clostridium difficile infection. Optionally, the method further comprises the step of obtaining a sample from a subject such as a human. The conditions suitable for binding include any set of conditions suitable or compatible with specific binding of an antigen and a cognate antibody or fragment thereof known in the art. In some embodiments, the antibody or antibody fragment is attached to a solid support, such as a glass or plastic chip or bead. In some embodiments of the detection method, the sample is a stool sample or a fluid exposed to a stool sample.

Another aspect of the disclosure is a method for diagnosing Clostridium difficile infection (CDI) in a subject comprising: (a) obtaining a biological sample from the subject; (b) contacting the sample with an antibody or antibody fragment as described above under conditions suitable for binding; (c) detecting the binding of the antibody or antibody fragment to Clostridium difficile toxin B, wherein the binding of the antibody or antibody fragment to toxin B is diagnostic of Clostridium difficile infection. Any set of conditions suitable or compatible with specific antigen-antibody binding that is known in the art is used. In some embodiments, the biological sample is a stool sample or a fluid exposed to a stool sample. One exemplary CDI is C. difficile-associated diarrhea.

Another aspect is drawn to a method for vaccinating a subject comprising administering an immunologically effective amount of the polypeptide according to the disclosure to a subject. An immunologically effective amount is that amount that will elicit a detectable immune response in the subject. In some embodiments of this aspect of the disclosure, the polypeptide comprises a sequence set forth in any of SEQ ID NOS:2-13.

Another aspect according to the disclosure is a method for diagnosing Clostridium difficile infection (CDI) in a subject comprising: (a) obtaining a biological sample from the subject; (b) adding to the sample a pair of PCR primers capable of amplifying a region of C. difficile tcdB between 8-750 nucleotides in length at the 3′ end of the tcdB coding region under polymerase chain reaction (PCR) conditions; (c) performing a PCR; and (d) diagnosing CDI if C. difficile tcdB is detected in the sample.

Another aspect is drawn to a method of preventing or treating Clostridium difficile Infection (CDI) comprising administering a therapeutically effective amount of the antibody or antibody fragment described herein to a subject, such as a human subject or patient. A related aspect of the disclosure provides a method of preventing or treating Clostridium difficile Infection (CDI) comprising administering a therapeutically effective amount of the polypeptide described herein, i.e., CDB-C250 or a fragment thereof containing at least one repeat element whose sequence is provided in FIG. 2 and wherein the polypeptide lacks the cytotoxic domain of toxin B. For each of these aspects, as for all of the methods of the disclosure, corresponding uses of the antibody, antibody fragment or polypeptide in the preparation of a medicament for detecting, diagnosing, treating or preventing CDI are contemplated.

Other features and advantages of the present disclosure will become apparent from the following detailed description. It should be understood, however, that the detailed description and the specific examples, while indicating preferred embodiments, are given by way of illustration only, because various changes and modifications within the spirit and scope of the invention will become apparent to those skilled in the art from the detailed description.

### BRIEF DESCRIPTION OF THE DRAWING

The following drawing forms part of the present specification and is included to further illustrate aspects of the disclosure. The disclosure may be better understood by reference to the figures of the drawing in combination with the detailed description of the specific embodiments presented herein.

FIG. 1 provides an alignment of the amino acid sequences of C. difficile toxin A and of C. difficile toxin B.

FIG. 2 provides the amino acid sequence (single-letter code) of a region containing sequence repeat motifs in the C-terminal region of 250 amino acids of C. difficile toxin B (C-250) identified in this disclosure as having effective immunogenicity.

FIG. 3 presents a panel of Western blots. Each blot contained purified CDB-C250, a crude lysate of a C. difficile (ATCC 9689) that produced reduced amounts of toxin and a crude lysate of a pathogenic strain of C. difficile (ATCC 43255) that is a strong toxin producer. Anti-CDB-C250 mAb probes were present in culture supernatants of hybridomas 1C11, 2C10, 3E1, 3G8, 3H10, and 4B3.

FIG. 4 provides the amino acid sequences of the variable regions of the heavy chain (upper panel) and light chain (lower panel) of anti-CDB-C250 monoclonal antibodies identified in Example 5. The complementarity determining regions are highlighted by identification as CDR1, CDR2 and CDR3 for each of the heavy and light chain variable regions. A horizontal bar over a region of the consensus sequence demarcates the consensus sequence of each CDR and a horizontal bar under regions of the monoclonal antibody sequences demarcates CDR regions in those antibodies.

### DETAILED DESCRIPTION

The exotoxins of Clostridium difficile, i.e., toxin A and toxin B, have long-confounded medical practitioners and researchers in the field of infectious disease and, in particular, infectious disease in the mammalian bowel, such as Clostridium difficile infection or CDI. The accepted view was that both toxin A and toxin B are important to the elaboration of CDI, but it is now realized that toxin B is the C. difficile virulence factor for CDI and is the more significant contributor to the CDI constellation of diseases (Lyras et al, Nature 458(7242):1176-9, 2009). With the historical background that assumed both toxins were important in disease, it is not surprising that some in the field were untroubled by the difficulty in experimentally or diagnostically distinguishing the presence of toxin A and the presence of toxin B by antibody methods. Importantly, C. difficile toxin A and toxin B share significant sequence similarities. Based on the protein structure analyses generally described herein, the C-terminal 250 amino acids of toxin B were identified as a segment unique to toxin B, with no homologous/similar counterpart in toxin A and thus formed the basis of the work, disclosed herein, to isolate this segment and use it for antibody preparation as well as to identify this segment as a source of vaccine polypeptides and therapeutic development in the management of CDI.

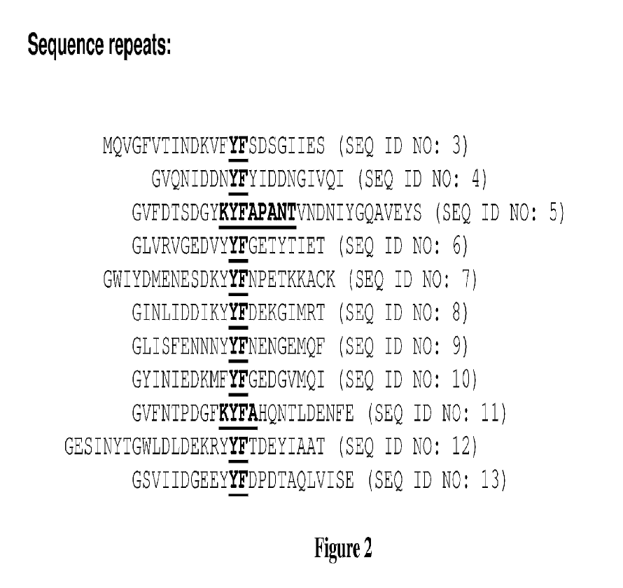

The disclosure establishes that the distinction between the detection of toxin A and the detection of toxin B is significant, with toxin B being the exotoxin whose presence is correlated with pathogenic C. difficile. The disclosure further provides compositions and methods for specifically detecting toxin B, providing a basis for the methods of diagnosing the presence of C. difficile and for diagnosing CDI, with these methods enjoying markedly improved accuracy and precision over methods known in the art. In particular, the disclosure provides expression vectors encoding toxin B fragments lacking the cytotoxic domain, such as the C-terminal 250-amino-acid fragment of toxin B (i.e., CDB-C250) or fragments thereof containing at least one of the repeat elements whose sequences are disclosed in FIG. 2 (SEQ ID NOS:3-13). The disclosure further provides that these vectors have been expressed in Escherichia coli and a purification methodology for CDB-C250 or fragments thereof is also provided. The purified CDB-C250 was used to elicit high-affinity monoclonal antibodies (mAbs). The monoclonal antibodies specifically recognizing the C-terminal domain (250 amino acids) of toxin B are contemplated for use in improved EIAs, such as ELISAs, useful in diagnosing CDI. In addition, the disclosure comprehends PCR-based assays that target the 3′ end of tcdB encoding part or all of CDB-C250, e.g., real-time PCR, to detect expression of tcdB, the gene encoding toxin B, as diagnostic methods for CDI. To aid in the detailed description of the compositions and methods according to the disclosure, a few express definitions are provided to facilitate an unambiguous disclosure of the various aspects of the disclosure.

### DEFINITIONS

The term “toxin A” refers to the toxin A protein encoded within the genome of C. difficile. The amino acid sequence of C. difficile toxin A is set forth in SEQ ID NO:14.

“Toxin B” refers to the toxin B protein encoded within the genome of C. difficile. The amino acid sequence of C. difficile toxin B is provided in SEQ ID NO: 1.

“CDB-C250” refers to the C-terminal 250-amino-acid region of C. difficile toxin B. The sequence of CDB-C250 is set forth in SEQ ID NO:2.

“CDI” means Clostridium difficile infection including, but not limited to, Clostridium difficile-associated diarrhea. CDI typically involves a disease of the gastrointestinal tract of a mammal, such as a human.

An “anti-C. difficile antibody” is an antibody that interacts with (e.g., specifically binds to) a protein or other component produced by a C. difficile bacterium. An “anti-toxin antibody” is an antibody that interacts with a toxin produced by C. difficile (e.g., toxin A or toxin B). An anti-toxin protein antibody may bind to an epitope, e.g., a conformational or a linear epitope, or to a fragment of the full-length toxin protein.

A “toxin B polypeptide lacking the cytotoxic domain” is a polypeptide fragment of C. difficile toxin B that is incapable of inducing cytotoxicity because the cytotoxic domain of the toxin B holo-protein is lacking. Exemplary toxin B polypeptides lacking the cytotoxic domain include the CDB-C250 toxin B fragment containing the C-terminal 250 amino acids of the protein, as well as fragments of the CDB-C250 region containing at least one of the repeat elements whose sequences are disclosed in FIG. 2. As shown in FIG. 2, these repeat elements are each about 20 amino acids in length.

The term “limit of detection” or “LOD” or “sensitivity” as used herein generally refers generally to the lowest analyte (e.g., toxin B, C-terminal fragment thereof, or C-terminal repeat-containing peptide thereof) concentration in a body fluid (e.g., serum) sample that can be detected but not necessarily quantitated as an exact value.

“Protein” is used interchangeably with “polypeptide.”

A “human antibody,” is an antibody that has variable and constant regions derived from human germline immunoglobulin sequences. The human antibodies described herein may include amino acid residues not encoded by human germline immunoglobulin sequences (e.g., mutations introduced by random or site-specific mutagenesis in vitro or by somatic mutation in vivo).

An anti-toxin B antibody, or antigen binding portion thereof, can be administered alone or in combination with a second agent. The subject can be a patient infected with C. difficile, or having a symptom of C. difficile infection (“CDI”; e.g., diarrhea, colitis, abdominal pain) or a predisposition towards C. difficile-associated disease (e.g., undergoing treatment with antibiotics, or having experienced C. difficile infection and at risk for relapse of the disease). The treatment can be to cure, heal, alleviate, relieve, alter, remedy, ameliorate, palliate, improve, or affect the infection and the disease associated with the infection, the symptoms of the disease, or the predisposition toward the disease.

An amount of an anti-toxin B antibody (or antibody fragment) or toxin B polypeptide lacking the cytotoxic domain that is effective to treat CDI, or a “therapeutically effective amount,” is an amount of the antibody (or fragment) or polypeptide that is effective, upon single or multiple dose administration to a subject, in inhibiting CDI in a subject. A therapeutically effective amount of the antibody (or antibody fragment) or polypeptide may vary according to factors such as the disease state, age, sex, and weight of the individual, and the ability of the antibody (or antibody fragment) or polypeptide to elicit a desired response in the individual. A therapeutically effective amount is also one in which any toxic or detrimental effects of the antibody (or antibody fragment) or polypeptide is outweighed by the therapeutically beneficial effects. The ability of an antibody (or fragment thereof) or polypeptide to inhibit a measurable parameter can be evaluated in an animal model system predictive of efficacy in humans. For example, the ability of an anti-toxin antibody to protect mice from lethal challenge with C. difficile can predict efficacy in humans. Other animal models are expected to be predictive of efficacy. Alternatively, this property of an antibody (or antibody fragment) or polypeptide can be evaluated by examining the ability of the compound to modulate at least one toxin B effect, such as modulation in vitro, by assays known to the skilled practitioner. In vitro assays include binding assays, such as ELISA, neutralization assays, and competitive inhibition assays.

An amount of an anti-toxin B antibody (or fragment thereof) or toxin B polypeptide lacking the cytotoxic domain that is effective to prevent a disorder, or a “a prophylactically effective amount,” is an amount that is effective, upon single- or multiple-dose administration to the subject, in preventing or delaying the occurrence or the onset or recurrence of CDI, or inhibiting a symptom thereof. If longer time intervals of protection are desired, however, increased doses can be administered.

The terms “agonize,” “induce,” “inhibit,” “potentiate,” “elevate,” “increase,” “decrease,” or the like, e.g., which denote quantitative differences between two states, refer to a difference, e.g., a statistically or clinically significant difference, between the two states.

As used herein, “specific binding” or “specifically binds to” refers to the ability of an antibody to: (1) bind to a toxin of C. difficile with an affinity of at least 1×10⁷ M⁻¹, and (2) bind to a toxin of C. difficile with an affinity that is at least two-fold greater than its affinity for a nonspecific antigen.

An “antibody” is given the broadest definition consistent with its meaning in the art, and includes proteins, polypeptides and peptides capable of binding to at least one binding partner, such as a proteinaceous or non-proteinaceous antigen. An “antibody” is a protein including at least one or two, heavy (H) chain variable regions (abbreviated herein as V_{H}), and at least one or two light (L) chain variable regions (abbreviated herein as V_{L}). The V_{H} and V_{L} regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al., J. Mol. Biol. 196:901-917, 1987, which are incorporated herein by reference). In some embodiments, each V_{H} and V_{L} is composed of three CDRs and four FRs, arranged from amino-terminus to carboxy-terminus in the following order: FR1, CDR1, FR2, CDR2, FR3, CDR3, FR4.

An “antibody” as used herein includes members of the immunoglobulin superfamily of proteins, of any species, of single- or multiple-chain composition, and variants, analogs, derivatives and fragments of such molecules. Specifically, an “antibody” includes any form of antibody known in the art, including but not limited to, monoclonal and polyclonal antibodies, chimeric antibodies, CDR-grafted antibodies, humanized antibodies, human antibodies, single-chain variable fragments, bi-specific antibodies, diabodies, and antibody fusions.

A “binding domain” is a peptide region, such as a fragment of a polypeptide derived from an immunoglobulin (e.g., an antibody), that specifically binds one or more specific binding partners. If a plurality of binding partners exists, those partners share binding determinants sufficient to detectably bind to the binding domain. In some embodiments, the binding domain is a contiguous sequence of amino acids.

An “epitope” is given its ordinary meaning herein of a single antigenic site, i.e., an antigenic determinant, on a substance (e.g., a protein) with which an antibody specifically interacts, for example by binding. Other terms that have acquired well-settled meanings in the immunoglobulin (e.g., antibody) art, such as a “variable light region,” variable heavy region,” “constant light region,” constant heavy region,” “antibody hinge region,” “complementarity determining region,” “framework region,” “antibody isotype,” “F_{C} region,” “constant region,” “single-chain variable fragment” or “scFv,” “diabody,” “chimera,” “CDR-grafted antibody,” “humanized antibody,” “shaped antibody,” “antibody fusion,” and the like, are each given those well-settled meanings known in the art, unless otherwise expressly noted herein.

The V_{H} or V_{L} chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region includes three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda.

“Immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon, and mu constant region genes, as well as the myriad immunoglobulin variable region genes. Full-length immunoglobulin “light chains” (about 25 KD and 214 amino acids) are encoded by a variable region gene at the NH₂-terminus (about 110 amino acids) and a kappa or lambda constant region gene at the COOH-terminus. Full-length immunoglobulin “heavy chains” (about 50 KD and 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having CDRs from a human or non-human source. The framework of the immunoglobulin can be human, humanized, or non-human, e.g., a murine framework modified to decrease antigenicity in humans, or a synthetic framework, e.g., a consensus sequence.

As used herein, “isotype” refers to the antibody class (e.g., IgM or IgG.sub.1) that is encoded by heavy chain constant region genes.

The term “antigen binding portion” of an antibody (or simply “antibody portion,” or “portion”), as used herein, refers to a portion of an antibody that specifically binds to a toxin of C. difficile (e.g., toxin B), e.g., a molecule in which one or more immunoglobulin chains is not full length, but which specifically binds to a toxin. Examples of binding portions encompassed within the term “antigen-binding portion” of an antibody include (i) a Fab fragment, a monovalent fragment consisting of the VLC, VHC, CL and CH1 domains; (ii) a F(ab′).sub.2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment (Ward et al., Nature 341:544-546, 1989), which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen binding portion” of an antibody. These antibody portions are obtained using conventional techniques known to those with skill in the art, and the portions are screened for utility in the same manner as are intact antibodies.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or portions thereof with a single molecular composition.

The term “recombinant” antibody, as used herein, refers to antibodies that are prepared, expressed, created, or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created, or isolated by any other means that involves splicing of human immunoglobulin gene sequences to other DNA sequences. Such recombinant antibodies include humanized, CDR grafted, chimeric, in vitro-generated (e.g., by phage display) antibodies, and may optionally include constant regions derived from human germline immunoglobulin sequences. Recombinant antibodies also include polypeptide products comprising at least one peptide corresponding to a part of an antibody, such as an Fv fragment, a single-chain antibody, a single-chain F_{V} (i.e., scFv) molecule, a linear antibody, a diabody, a peptibody, a bi-body (bispecific Fab-scFv), a tribody (Fab-(scFv)2), a hinged or hingeless minibody, a mono- or bi-specific antibody, or an antibody fusion. A peptide corresponds to a part of an antibody if it has a primary amino acid sequence at least 95% identical to a part of an antibody or if it contains at least one domain recognizable by one of skill in the art as an antibody domain. Peptide linkers of about 10-100 amino acids are used where appropriate to link polypeptide domains of a recombinant antibody, as would be known in the art.

As used herein, the term “substantially identical” (or “substantially homologous”) refers to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the first antibody. Calculations of “homology” between two sequences are performed as described in Example 2 and such calculations are known in the art.

It is understood that the antibodies and antigen binding portions thereof described herein may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie et al., Science, 247:1306-1310, 1990. A “conservative amino acid substitution” is one in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of a polypeptide, such as a binding agent, e.g., an antibody, without substantially altering a biological activity, whereas an “essential” amino acid residue results in such a change.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. 6.3.1-6.3.6, 1989, which is incorporated herein by reference. Aqueous and nonaqueous methods are described in that reference and either can be used. Specific hybridization conditions referred to herein are as follows: 1) low stringency hybridization conditions: 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions: 6×SSC at about 45° C., followed by one or more washes in 0.2×SSC, 0.1% SDS at 60° C.; 3) high stringency hybridization conditions: 6×SSC at about 45° C., followed by one or more washes in 0.2×SSC, 0.1% SDS at 65° C.; and 4) very high stringency hybridization conditions: 0.5 M sodium phosphate, 7% SDS at 65° C., followed by one or more washes at 0.2×SSC, 1% SDS at 65° C.

Unless otherwise defined, all technical and scientific terms used herein have the meaning commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

C. difficile is a Gram-positive, toxin-producing bacterium that causes antibiotic-associated diarrhea and colitis in humans. Provided herein are methods and compositions for treatment and prevention of C. difficile infection/disease (CDI). The compositions include antibodies and antibody fragments that specifically recognize or bind to the C-terminal domain (250 amino acids) of toxin B of C. difficile. In particular, human monoclonal antibodies are provided. In certain embodiments, these human monoclonal antibodies are produced in mice expressing human immunoglobulin gene segments (described below). Combinations of anti-toxin B antibodies are also provided, as are any form of recombinant antibody or antibody fragment specifically recognizing C. difficile toxin B, such as the C-terminal 250-amino-acid region of intact toxin B, regardless of whether such region is found in intact toxin B or a fragment thereof.

The methods according to the disclosure include contacting a biological sample with an anti-toxin B-specific antibody or antigen-binding portion under conditions suitable for binding and diagnosing C. difficile infection and/or CDI on the basis of the binding detected. Additional methods according to the disclosure comprise administering to a subject an antibody (or antigen-binding portion thereof), or a plurality of antibodies and/or antigen-binding portions thereof, that bind to C. difficile toxin B to inhibit or prevent CDI in the subject. For example, human monoclonal anti-toxin B antibodies described herein can neutralize toxin B and inhibit relapse of C. difficile-mediated disease. In other examples, combinations of anti-toxin B antibodies (e.g., anti-toxin B monoclonal antibodies) can be administered to inhibit primary disease and reduce the incidence of disease relapse. The human monoclonal antibodies may localize to sites of disease (e.g., the gut) in vivo. In still other examples, a polypeptide comprising the C-terminal 250-amino-acid region of C. difficile toxin B or any of the repeat sequences identified in SEQ ID NOS:3-13 is administered to inhibit primary disease and to reduce the incidence of disease relapse as well as being used in vaccine production to inhibit primary disease and to reduce the incidence of disease relapse. The C-terminal 250-amino-acid region of C. difficile toxin B fragment, or polypeptides comprising any one or more of the repeat sequences of SEQ ID NOS:3-13 are useful to administer so that the polypeptide can localize to cell-binding sites of C. difficile toxin (e.g., the gut) to prevent disease in vivo, while minimizing the deleterious effects associated with administering intact toxin B.

In general, animals are immunized with antigens expressed by C. difficile to produce antibodies. For producing anti-toxin antibodies, what had been known in the art was immunization with inactivated toxins, or toxoids. Toxins can be inactivated, e.g., by treatment with formaldehyde, SDS, glutaraldehyde, peroxide, or oxygen treatment. Mutant C. difficile toxins with reduced toxicity can be produced using recombinant methods (see, e.g., U.S. Pat. Nos. 5,085,862; 5,221,618; 5,244,657; 5,332,583; 5,358,868; and 5,433,945). For example, mutants containing deletions or point mutations in the toxin active site can be made. Recombinant fragments of the toxins can be used as immunogens. These techniques, however, result in the use of immunogens that differ from the desired target of any elicited antibody. Another approach is to inactivate the toxin by treatment with UDP-dialdehyde. This approach also results in immunogens that differ from the target of the elicited antibody. Disclosed herein is an advance in methods of producing anti-toxin B-specific antibodies comprising the use of an immunogen derived from the C-terminal 250-amino-acid polypeptide of C. difficile toxin B in a native form. That is, the polypeptide comprising the C-terminal 250-amino-acid region of toxin B and/or the intact toxin B from which the C-250 fragment may be physically derived, are not used as immunogens or sources of immunogens in a denatured or otherwise inactivated form.

The antibodies of the present invention are said to be immunospecific or specifically binding if they bind to antigen with a Kₐ of greater than or equal to about 10⁴M⁻¹, 10⁵M⁻¹, 10⁶M⁻¹, 10⁷M⁻¹, 10⁸M⁻¹, 10⁹M⁻¹, or 10¹⁰M⁻¹. The anti-toxin B antibodies bind to different naturally occurring forms of C. difficile toxin B, including intact toxin B and fragments thereof. The monoclonal antibodies disclosed herein have affinity for the C-terminal 250-amino-acid portion of C. difficile toxin B and are characterized by a dissociation equilibrium constant (Kd) of at least about 10⁻⁴ M, at least about 10⁻⁷ M, at least about 10⁻⁸ M, at least about 10⁻¹⁰ M, at least about 10⁻¹¹ M, or at least about 10⁻¹² M. Monoclonal antibodies and antigen-binding fragments thereof that are suitable for use in the methods of the disclosure are capable of specifically binding to toxin B. Such affinities may be readily determined using conventional techniques, such as by equilibrium dialysis; by using the BIAcore 2000 instrument, using general procedures outlined by the manufacturer; by radioimmunoas say using ¹²⁵I labeled toxin B; or by other methods known in the art. The affinity data is analyzed, for example, by the method of Scatchard et al., Ann N.Y. Acad. Sci., 51:660 (1949). Thus, it will be apparent that preferred toxin B antagonists will exhibit a high degree of specificity for toxin B and will bind with substantially lower affinity to other molecules, including C. difficile toxin A.

The antigen to be used for production of antibodies is, e.g., intact toxin B, a C-terminal fragment of toxin B of 250 amino acids (i.e., CDB-C250), or a fragment of CDB-C250 containing at least one repeat element from CDB-C250, is optionally fused to another polypeptide that facilitates epitope display.

Polyclonal antibodies are raised in animals by multiple subcutaneous (sc) or intraperitoneal (ip) injections of, e.g., the CDB-C250 fragment of toxin B and an adjuvant. An improved antibody response may be obtained by conjugating, e.g., CDB-C250 to a protein that is immunogenic in the species to be immunized, e.g., keyhole limpet hemocyanin, serum albumin, bovine thyroglobulin, or soybean trypsin inhibitor, using a bifunctional or derivatizing agent such as maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with one fifth to one tenth the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. The animal is typically boosted with the conjugate of the same antigen, but conjugated to a different protein and/or through a different cross-linking reagent are contemplated. Conjugates also can be made in recombinant cell culture as protein fusions. Also, aggregating agents such as alum are suitably used to enhance the immune response.

Monoclonal antibodies are made using, e.g., the hybridoma method first described by Kohler et al., Nature, 256:495 (1975), or by recombinant DNA methods. In the hybridoma method, a mouse or other appropriate host animal, such as a hamster or macaque monkey, is immunized to elicit lymphocytes that produce or are capable of producing antibodies that will specifically bind to the protein, e.g., CDB-C250, used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), because those substances prevent the growth of HGPRT-deficient cells.

Exemplary myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. The binding specificity of monoclonal antibodies produced by hybridoma cells is determined, e.g., by immunoprecipitation or by an in vitro binding assay, such as a radioimmunoas say (RIA) or an enzyme-linked immunosorbent assay (ELISA). The binding affinity of the monoclonal antibody are, for example, determined by Scatchard analysis (Munson et al., Anal. Biochem., 107:220 (1980)).

After hybridoma cells are identified that produce antibodies of the desired specificity, affinity, and/or activity, the clones are subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

An antigen-antibody reaction is described in this paragraph in the context of immobilized antigen interacting with free antibody in an ELISA embodiment. Initially, 96-well plates are coated overnight at 48° C. with 100 μL per well of toxin B (25 μg/mL) in carbonate-bicarbonate buffer 50 mM, pH 9.6 (Sigma-Aldrich, St Louis, Mo.). Antibody preparations are diluted appropriately, e.g., 1:50 to 1:20, in 2% BSA, 0.05% Tween phosphate buffer saline (PBST), as would be known in the art. Diluted antibody is then added to the plate and incubated for two hours at room temperature. Toxin B-specific antibodies are detected with horseradish peroxidase-conjugated goat secondary antibody (KPL, Gaithersburg, Md.) diluted, e.g., 1:2500. The immobilized horseradish peroxidase is then revealed by adding tetramethylbenzidine peroxidase substrate (KPL) to the wells, and results are obtained using a microplate reader at 650 nm.

DNA encoding the monoclonal antibodies are also contemplated by the disclosure and may be isolated and sequenced from the hybridoma cells using conventional procedures (e.g., by using oligonucleotide probes that are capable of binding specifically to genes encoding the heavy and light chains of the monoclonal antibodies). Once isolated, the DNA may be recombined in expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies is well known in the art.

Although these standard techniques are known, it is desirable to use humanized or human antibodies rather than murine antibodies to treat human subjects, because humans mount an immune response to antibodies from mice and other species. The immune response to murine antibodies is called a human anti-mouse antibody or HAMA response and is a condition that causes serum sickness in humans and results in rapid clearance of the murine antibodies from the circulation of an individual. The immune response in humans has been shown to be against both the variable and the constant regions of murine immunoglobulins. Human monoclonal antibodies are safer for administration to humans than antibodies derived from other animals and human polyclonal antibodies.

One type of animal useful in generating human monoclonal antibodies is a transgenic mouse that expresses human immunoglobulin genes rather than its own mouse immunoglobulin genes. Such transgenic mice, e.g., “HuMAb™” mice, contain human immunoglobulin gene miniloci that encode unrearranged human heavy (.mu. and .gamma.) and .kappa. light chain immunoglobulin sequences, together with targeted mutations that inactivate the endogenous μ and κ chain loci (see, e.g., Lonberg, et al., Nature 368(6474): 856-859, 1994, and U.S. Pat. No. 5,770,429). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, et al., supra; reviewed in Lonberg, N. Handbook of Experimental Pharmacology 113:49-101, 1994; Lonberg, et al., Intern. Rev. Immunol., 13: 65-93, 1995, and Harding, et al., Ann. N.Y. Acad. Sci., 764:536-546, 1995).

The preparation of such transgenic mice is described in further detail in Taylor, et al., Nucl. Acids Res., 20:6287-6295, 1992; Chen, et al., Internl. Immunol. 5: 647-656, 1993; Tuaillon et al., Proc. Natl. Acad. Sci. (USA) 90:3720-3724, 1993; Choi et al., Nature Genetics, 4:117-123, 1993; Chen, et al, EMBO J., 12: 821-830, 1993; Tuaillon et al., J. Immunol., 152:2912-2920, 1994; Taylor, et al., Internl. Immunol., 6: 579-591, 1994; and Fishwild, et al., Nature Biotechnology, 14: 845-851, 1996. See also, U.S. Pat. Nos. 5,545,806; 5,569,825, 5,625,126, 5,633,425, 5,661,016, 5,770,429, 5,789,650, 5,814,318, 5,874,299 and 5,877,397, and PCT Publication Nos. WO 01/14424, WO 98/24884, WO 94/25585, WO 93/1227, and WO 92/03918.

To generate fully human monoclonal antibodies to an antigen, HuMAb mice can be immunized with an immunogen, as described by Lonberg, et al. Nature, 368(6474): 856-859, 1994; Fishwild, et al., Nature Biotechnology, 14: 845-851, 1996 and WO 98/24884. The mice are 6-16 weeks of age upon the first immunization. For example, a purified preparation of the peptide containing the C-terminal 250 amino acids of toxin B can be used to immunize the HuMAb mice intraperitoneally.

HuMAb transgenic mice respond best when initially immunized intraperitoneally (IP) with antigen in complete Freund's adjuvant, followed by IP immunizations every other week (up to a total of 6) with antigen in incomplete Freund's adjuvant. The immune response can be monitored over the course of the immunization protocol with plasma samples being obtained by retro-orbital bleeds. The plasma can be screened, for example by ELISA or flow cytometry, and mice with sufficient titers of anti-toxin human immunoglobulin are used for fusions. Mice are optionally boosted intravenously with antigen 3 days before sacrifice and removal of the spleen. It is expected that 2-3 fusions for each antigen may need to be performed.

The mouse splenocytes can be isolated and fused with PEG to a mouse myeloma cell line based upon standard protocols. The resulting hybridomas are then screened for the production of antigen-specific antibodies. For example, single cell suspensions of splenic lymphocytes from immunized mice are fused to one-sixth the number of P3×63-Ag8.653 or other nonsecreting mouse myeloma cells (ATCC, CRL 1580) with 50% PEG. Cells are plated at approximately 2×10⁴ in flat-bottom microtiter plates, followed by a two-week incubation in selective medium containing 20% fetal clone serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamicin and 1×HAT medium (Sigma; the HAT is added 24 hours after the fusion). After two weeks, cells are cultured in medium in which the HAT is replaced with HT. Supernatants from individual wells are then screened by ELISA for human anti-toxin B monoclonal IgM and IgG antibodies. The antibody-secreting hybridomas are replated, screened again, and if still positive for human IgG, anti-toxin monoclonal antibodies, can be subcloned at least twice by limiting dilution. The stable subclones are then cultured in vitro to generate small amounts of antibody in tissue culture medium for characterization.

The repertoire in the transgenic mouse will ideally approximate that shown in a non-transgenic mouse, usually at least about 10% as high, preferably 25 to 50% or more as high. Generally, at least about a thousand different immunoglobulins (ideally IgG), preferably 10⁴ to 10⁶ or more, will be produced, depending primarily on the number of different V, J, and D regions introduced into the mouse genome. Typically, the immunoglobulins will exhibit an affinity for preselected antigens of at least about 10⁷ M⁻¹, 10⁹ M⁻¹, 10¹⁰ M⁻¹, 10¹¹ M⁻¹, 10¹²M⁻¹, or greater, e.g., up to 10¹³ M⁻¹ or greater.

HuMAb mice can produce B cells that undergo class-switching via intra-transgene switch recombination (cis-switching) and express immunoglobulins reactive with the toxin. The immunoglobulins can be human sequence antibodies, wherein the heavy and light chain polypeptides are encoded by human transgene sequences, which may include sequences derived by somatic mutation and V region recombined joints, as well as germline-encoded sequences. These human sequence immunoglobulins can be referred to as being effectively identical to a polypeptide sequence encoded by a human VL or VH gene segment and a human JL or JH segment, even though other non-germline sequences may be present as a result of somatic mutation and differential V-J and V-D-J recombination joints. With respect to such human sequence antibodies, the variable regions of each chain are typically at least 80 percent encoded by human germline V, J, and, in the case of heavy chains, D gene segments. Frequently at least 85 percent of the variable regions are encoded by human germline sequences present on the transgene. Often 90 or 95 percent or more of the variable region sequences are encoded by human germline sequences present on the transgene. However, since non-germline sequences are introduced by somatic mutation and VJ and VDJ joining, the human sequence antibodies will frequently have some variable region sequences (and less frequently constant region sequences) that are not encoded by human V, D, or J gene segments as found in the human transgene(s) in the germline of the mice. Typically, such non-germline sequences (or individual nucleotide positions) will cluster in or near CDRs, or in regions where somatic mutations are known to cluster.

The human sequence antibodies that bind to toxin B can result from isotype switching, such that human antibodies comprising a human sequence gamma chain (such as gamma 1, gamma 2, or gamma 3) and a human sequence light chain (such as κ) are produced. Such isotype-switched human sequence antibodies often contain one or more somatic mutation(s), typically in the variable region and often in or within about 10 residues of a CDR, as a result of affinity maturation and selection of B cells by antigen, particularly subsequent to secondary (or subsequent) antigen challenge. These high-affinity human sequence antibodies have binding affinities of at least about 1×10⁹ M⁻¹, typically at least 5×10⁹ M⁻¹, frequently more than 1×10¹⁰M⁻¹, and sometimes 5×10¹⁰ M⁻¹ to 1×10¹¹ M⁻¹ or greater. Anti-toxin antibodies can also be raised in other animals, including but not limited to non-transgenic mice, humans, rabbits, goats, and chicken.

The following examples illustrate embodiments of the invention. Example 1 describes the cloning of a polynucleotide encoding CDB-C250 and the expression of that polypeptide. Example 2 discloses a comparison of the full-length amino acid sequences of C. difficile toxin A and C. difficile toxin B. Example 3 provides a characterization of CDB-C250, the C-terminal 250-amino-acid region of C. difficile toxin B. Example 4 describes the elicitation of antibodies specifically recognizing C. difficile toxin B, and not detectably recognizing C. difficile toxin A. Example 5 provides the results of Western blot analyses establishing the specificity of anti-toxin B antibodies according to the disclosure. Example 6 discloses an in vitro cytotoxicity assay for toxin B, CDB-C250 and other toxin B fragments. Examples 7 and 8 provide in vivo animal models for assessing the effects of C. difficile infection as well as the effects of prophylactics and/or therapeutics therefor. Example 9 discloses further uses of antibodies in measuring C. difficile toxin B and fragments thereof such as CDB-C250 and peptides comprising at least one repeat motif from the C-terminal 250-amino-acid domain. Example 10 discloses methods used to maintain and confirm the identities of various C. difficile species used in the studies.

### Example 1

### Cloning and Expression of Toxin B and CDB-C250

C. difficile toxin A and toxin B share significant sequence similarities, which is the primary reason that past attempts to develop high-affinity antibody directed against toxin B (for use in diagnostic tests) have failed. A comparison of the amino acid sequences of C. difficile toxin A and C. difficile toxin B was performed, as described in Example 2. Based on protein structure analysis, the C-terminal 250 amino acids of toxin B (CDB-C250) were identified as a segment unique to toxin B, with no similar counterpart in toxin A.

The coding region for toxin B was obtained using conventional cloning technologies. Initially, genomic DNA was extracted from C. difficile strain ATCC 43255 (a strong toxin B-producing isolate). PCR was then used to amplify the toxin B coding region using the extracted genomic DNA as template. Amplified products were cloned and a DNA encoding the C-terminal 250 amino acids of toxin B was identified. This DNA fragment was then cloned into a prokaryotic expression plasmid (pAED4) for protein expression in E. coli. More particularly, the DNA encoding CDB-C250 was cloned in the T7 RNA polymerase-based expression plasmid pAED4 and the resulting clone was used to transform BL21(DE3)pLysS E. coli cells. Freshly transformed bacteria were cultured in 2× tryptone-yeast broth containing ampicillin and chloramphenicol at 37° C. with vigorous shaking. The culture was induced during log-phase of growth with 0.4 mM isopropyl-1-thio-β-D-galactopyranoside. After 3 additional hours of culture, the bacterial cells were harvested by centrifugation and lysed by three passages through a French Press. The bacterial lysate was fractionated by ammonium sulfate precipitation, dialyzed and separated on a DE52 anion exchange column in 6 M urea at pH 7.0. The CDB-C250 peak identified by SDS-PAGE was dialyzed and concentrated by lyophilization for further purification on a Sephadex G-75 gel filtration column at pH 7.0 in the presence of 6 M urea, 0.5 M KCl and 0.1 mM EDTA. The purified CDB-C250 peak was identified by SDS-PAGE, dialyzed to remove urea and salt, and lyophilized. The CDB-C250 protein expressed from the clone showed very high level expression in E. coli, indicating excellent compatibility with the host bacterium. The CDB-C250 clone provided a ready reagent for producing CDB-C250 in quantity in any of a variety of in vivo, or in vitro, contexts.

In view of the success of the clone encoding CDB-C250 to express robust levels of CDB-C250, and the unique antigenicity of CDB-C250 demonstrated hereinbelow, it is expected that polynucleotides comprising the coding region for CDB-C250, or a fragment thereof, such as a polynucleotide encoding at least 6, 7, 8, 9, 10, 15, 20, 25, 30, 40, 50, 60, 70, 80, 90, 100, 125, 150, 175, 200 or 225 amino acids, or a polynucleotide encoding an amino acid sequence of length equal to any whole integer between 6 and 250 amino acids, will be useful in producing CDB-C250 or a fragment (6-250 amino acids from the CDB-C250 region) thereof for use as a prophylactic or for use as a therapeutic in preventing or treating CDI. In particular, it is expected that polynucleotides encoding at least one of the eleven repeat elements (about 20 amino acids in length) are useful in producing, via expression, a polypeptide that will competitively inhibit the cytotoxigenic activity of C. difficile toxin B and/or in serving as probes for nucleic-acid-based diagnostic assays for C. difficile as the causative agent of CDI.

An effective practical approach to deliver a polypeptide according to the disclosure to the colon will be an important step in animal treatment for CDI. In humans, and in larger domesticated animals such as cattle, horses, goats, sheep, cats, dogs and the like, it may be delivered via encapsulated capsules. Administration by oral capsule would be difficult, if not impossible, in mice and hamsters, and may prove unwieldy or undesirable for larger non-human animals. As an alternative, an approach relying on an engineered form of secreting Lactococcus lactis is used as a cell factory for in situ treatment of disease in the colon. L. lactis is a non-pathogenic, non-invasive, non-colonizing Gram-positive bacterium, mainly used to produce fermented foods. Recombinant L. lactis strains are known to be safe and effective for the production and in vivo delivery of cytokines. The use of engineered L. lactis secreting interleukin-10 for the treatment of inflammatory bowel disease has rapidly moved to clinical trials. As a gram-positive bacterium, L. lactis has only one cellular membrane. This makes it an ideal host for protein secretion with subsequent membrane- or cell-wall-anchoring, or export into the fermentation medium. Another advantage is the low extracellular proteinase activity in lactococci.

The development of this safe system for in vivo delivery of biologically active proteins/peptides as therapeutic agents is suitable for the use of CDB-C250, or CDB-C250 fragments containing a repeat element, to treat CDI, particularly in animals such as humans. Using the commercially available pNZ expression plasmid vectors, DNA clones encoding CDB-C250 (and/or CDB-C250 fragments containing at least one repeat element shown in FIG. 2) are constructed for expressing and secreting these products. Since codon usage was found to be an important factor in the efficiency of expressing exogenous genes in L. lactis whose genomic DNA has a GC content of 35-37%, the various coding fragments will need to be engineered using synthetic nucleotides. DNA coding templates of this length have been engineered by us using nested sets of multiple pairs of synthetic nucleotides of about 150 nucleotides each. After chain extension reactions in a thermocycler to generate three double-stranded DNA fragments with overlapping end sequences (about 280 bp each), two rounds of recombinant PCR will be carried out to join them into the 753-bp DNA coding template of CDB-C250, along with an NcoI cloning site at the 5′ end of the coding region for CDB-C250 and a couple restriction cloning sites at the 3′ end. To ensure sequence authenticity, the synthetic nucleotides are ordered as gel-purified full-length products.

PCR procedures are performed with proofreading polymerase and the final DNA insert constructed in the recombinant expression plasmids will be sequenced. During the cloning process, chloramphenicol sensitive, rec A⁺ strain of E. coli, such as MC1061, is used for the expression system. The CDB-C250 protein expression using the recombinant pNZ vectors is first carried out in L. lactis for protein purification and in vitro characterization. The transformation of L. lactis is accomplished using electroporation. Transformed cells will be examined for molecular weight, isoelectric point and Western blotting using anti-CDB-C250 monoclonal antibodies for authenticity. Large-scale expression will be performed following the instructions in the operating manual of the easy-to-operate and strictly controlled NICE® system (Bocascientific). The purification of CDB-C250 will be carried out as described above. It is worth noting that in comparison to the aerobically growing B. subtilis, which can secrete several grams of protein per liter, protein secretion in Lactococcus spp. is less substantial. This lower level of expression, however, is sufficient for testing relatively large-scale production of CDB-C250 in culture and therapeutic activity in vivo. To engineer a L. lactis strain that secretes CDB-C250 in the colon, the non-fusion CDB-C250-expressing pNZ vector is modified by adding the 27-amino-acid signal peptide of the major lactococcal-secreted protein Usp45 as a fusion peptide to the N-terminus of CDB-C250.

L. lactis expression and secretion of CDB-C250 protein is achieved in vivo in the mouse colon by administering to C57BL/6 mice, by intragastric inocula typically a daily dose (5-7 days total) of approximately 2×10⁷ colony forming units of transformed L. lactis. The L. lactis is transformed with the recombinant pNZ plasmid or control L. lactis is transformed with the pNZ empty vector and/or heat-killed L. lactis control expressing CDB-C250. Three, five and seven days after the final dose, mice are euthanized (sodium pentobarbital or secobarbital) and the colon contents extracted for SDS-PAGE and Western blotting analysis of CDB-C250 using anti-CDB-C250 monoclonal antibodies to validate the delivery of CDB-C250 to the mouse colon. In this way, the quantitative level of production and the integrity of the CDB-C250 protein produced in situ in mouse colon is evaluated. When necessary, longer incubation times in the mice before C. difficile challenge as well as additional doses of L. lactis inoculation are examined. Use of L. lactis to deliver polynucleotides encoding polypeptides according to the disclosure in a subject, such as a human, is contemplated as useful to prevent or treat CDI.

In addition to the foregoing discussion of polynucleotides encoding toxin B fragments or specifically hybridizing under stringent conditions thereto, the disclosure contemplates any pair of nucleic acid primers capable of specifically amplifying a 3′ region of tcdB. The tcdB gene encodes C. difficile toxin B. Suitable primers amplify the 3′ region of tcdB encoding CDB-C250 or a fragment thereof, and the targeted amplification of the 3′ end of tcdB as useful in diagnostic assays for the presence of C. difficile, as well as being useful in methods of producing a polynucleotide encoding CDB-C250 or a fragment thereof. The primer pairs according to the disclosure will specifically hybridize to DNA targets, preferably through complete complementarity. The DNA targets the primer pairs are offset from each other by about 18-750 nucleotides, or more, provided that any amplified nucleic acid product containing the sequence between the two targets is capable of specifically hybridizing to the 3′ region of C. difficile tcdB.

### Example 2

### Comparison of Amino Acid Sequences of Toxin a and Toxin B

A comparison of the amino acid sequences of C. difficile toxin A and C. difficile toxin B was performed in view of the known problem (see, e.g., U.S. Patent Publication No. 20050287150) of cross-reactivities of binding partners to either of these two exotoxins of C. difficile. The sequences were aligned to optimize similarity (i.e., gaps were introduced). In general, the length of a reference sequence aligned for comparison purposes is at least 50% of the length of that reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. The percent homology between two amino acid sequences is determined using the Needleman and Wunsch, J. Mol. Biol. 48:444-453, 1970, algorithm which has been incorporated into the GAP program in the GCG software package, using a Blossum 62 scoring matrix with a gap penalty of 12, a gap extend penalty of 4, and a frameshift gap penalty of 5.

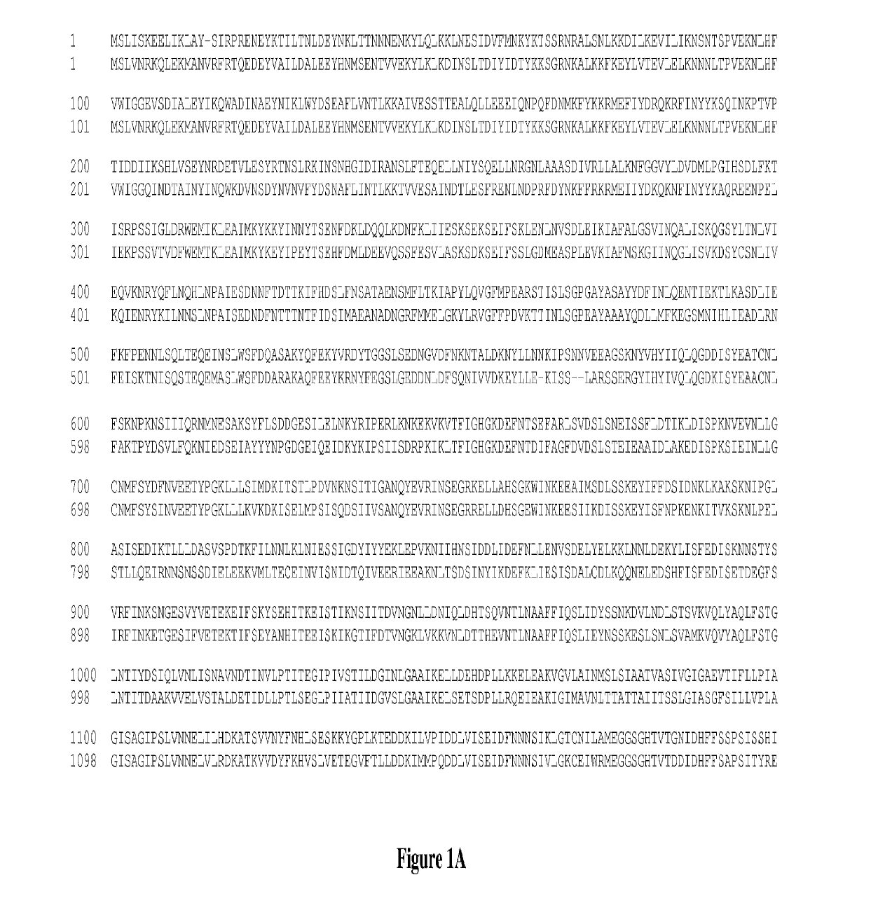

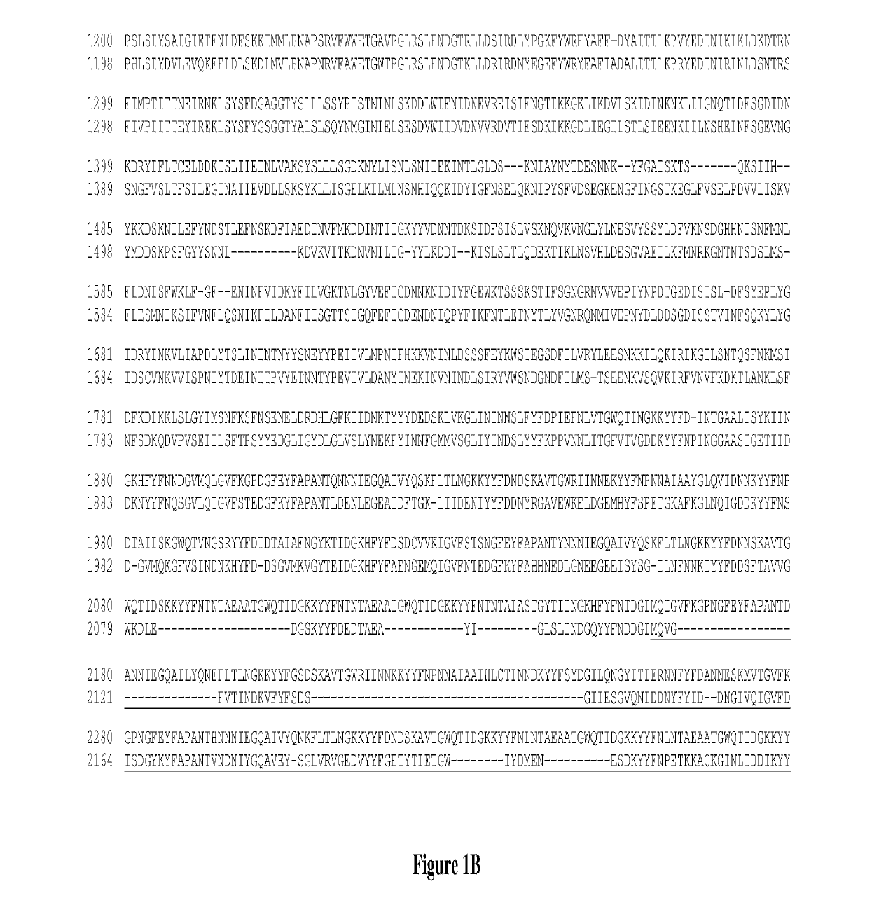

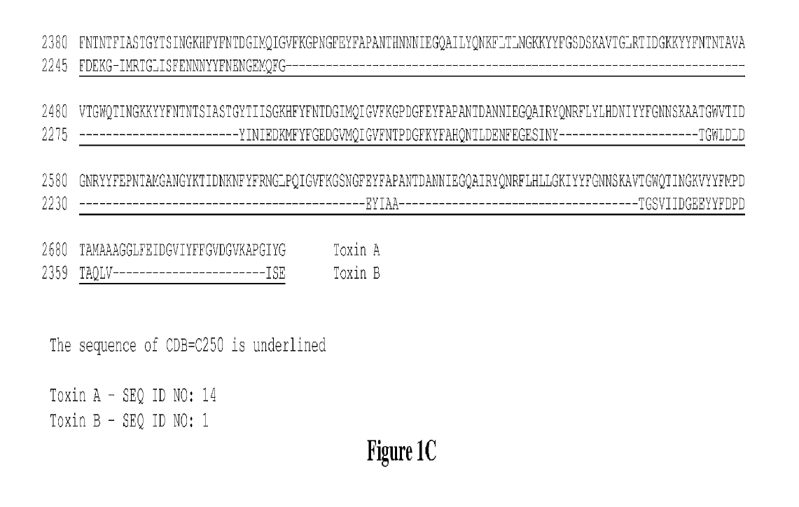

A comparison of the amino acid sequences of C. difficile toxin A and C. difficile toxin B is shown in FIG. 1. Sequence comparisons were performed using the Clustal method in the Megalign program from DNAStar. The repeat motifs were identified and aligned manually based on amino acid identities. The Figure shows divergence at the C-terminal ends of the amino acid sequences, and the C-terminal region of 250 amino acids of C. difficile toxin B (i.e., CDB-C250) was identified as the region within which toxin B-specific epitopes are located, i.e., epitopes unique to toxin B and not shared with toxin A. Also apparent in FIG. 1 is that an antibody recognizing an epitope in the aligned C-terminal region of 589 amino acids of toxin A would include regions of toxin B showing considerable similarity to toxin A, thereby producing a likely result of cross-reacting antibodies.

Continued analysis of the amino acid sequence of the C-terminal 250-amino-acid region of C. difficile toxin B revealed several amino acid repeat structures expected to form toxin B-specific epitopes, and to participate in toxin B-specific epitopes. The amino acid sequences of these repeat sequences are presented in FIG. 2. Aligning the repeat sequences manually based on amino acid identities, eleven repeats of about 20 amino acids per repeat were identified (FIG. 2). The gross mapping of the functional domains of toxins A and B indicated that the C-terminal regions of both toxins contain the cell surface receptor binding site. This domain outlined for toxin B contains about 500 amino acids and only the C-terminal half, i.e., the CDB-C250 region, contains the repeating motifs (FIG. 2). In contrast to the cellular receptor in the C-terminal domain of toxin A, which has been extensively studied (Jank et al., Glycobiol. 17:15R-22R (2007)), very little is known for the cellular receptor domain of toxin B in colon epithelial cells. Consistent with the observation that toxin A and toxin B target different cell surface receptors (Stubbe et al., J. Immunol. 164:1952-1960 (2000)), their sequences in the C-terminal domains are significantly different (FIG. 1). This notion is further supported by the observation that multiple anti-CDB-C250 monoclonal antibodies do not cross react with toxin A. These features support our expectation that CDB-C250, and peptides comprising at least one of the repeat structures of the C-terminal 250-amino acid domain, are benign competitors of toxin B useful in the treatment and prevention of CDI. Moreover, binding partners, e.g., antibodies or antibody fragments, that specifically recognize or bind one or more peptides comprising at least one repeat structure of FIG. 2 are expected to be useful in detecting the presence of C. difficile toxin B and, thereby, to be useful in diagnosing, preventing, or treating CDI.

### Example 3

### Characterization of CDB-C250

The cloned CBD-C250 protein showed very high level expression in E. coli, indicating excellent compatibility with the host bacterium. The purified CDB-C250 protein is highly soluble in physiological buffers as well as in water. From the unique amino acid sequence and the physicochemical properties of CBD-C250, it is apparent that this polypeptide is not only consistent with a toxin B-specific antigenic epitope comprising a plurality of smaller antigenic peptide sequences but also has properties indicative of therapeutic agent useful in countering the pathogenic effect of native toxin B, the exotoxin whose presence is correlated with CDI. Data disclosed in the following examples confirms that CDB-C250 is antigenic and is useful in diagnosing, preventing, and/or treating CDI in that it can elicit anti-toxin B-specific antibodies and can function itself as a toxin B competitor.

Various physicochemical analyses of the C-terminal 250-amino-acid region of C. difficile toxin B were undertaken using accepted, conventional techniques. The molecular weight, isoelectric point, pH-charge titration curves and hydrophilicity profile were analyzed with DNAStar software. The molecular weight and pH-charge relationship were verified by SDS-PAGE and ion-exchange chromatography. The primary amino acid sequence of the CDB-C250 polypeptide is set forth in SEQ ID NO:2; the primary amino acid sequence of intact toxin B is set forth in SEQ ID NO:1. The molecular weight of the C-terminal polypeptide comprising the 250 C-terminal residues of C. difficile toxin B was determined to be 29,000 daltons. This polypeptide has 14 strongly basic amino acids (Lys, Arg), 47 strongly acidic amino acids (Asp, Glu), 75 hydrophobic amino acids (Ala, Ile, Leu, Phe, Trp, and Val), and 79 polar amino acids (Asn, Cys, Gln, Ser, Thr, and Tyr). The isoelectric point of the C-terminal 250-amino-acid polypeptide of C. difficile toxin B is 3.722. Considering the primary amino acid sequence and the physicochemical properties of the C-terminal polypeptide, it is apparent that this polypeptide is not only consistent with an antigenic polypeptide, but with a polypeptide comprising a plurality of smaller peptide sequences that are antigenic, such as the repeat structures identified in FIG. 2 and addressed in Example 2.

Peptide fragments of CDB-C250, including fragments containing at least one repeat element from the CDB-C250 region (repeat element sequences are shown in FIG. 2 and provided in SEQ ID NOS:3-13), are expected to be useful in eliciting specific anti-toxin B antibodies and in competing with intact toxin B in prophylactic and therapeutic methods according to the disclosure.

### Example 4

### Elicitation of Monoclonal Antibodies Specific to Toxin B

Monoclonal antibodies in accordance with the disclosure were made by the hybridoma method first described by Kohler et al., (Nature, 256:495-7, 1975). Other methods of eliciting or generating mAbs are known in the art and may be used in preparing mAbs that specifically bind the C-terminal 250-amino-acid polypeptide of toxin B or a peptide thereof comprising a repeat sequence as set forth in FIG. 2. An exemplary alternative method for generating the antibodies is by recombinant DNA methods (see, e.g., U.S. Pat. No. 4,816,567). The monoclonal antibodies may also be isolated from phage antibody libraries using the techniques described in, for example, Marks et al., J. Mol. Biol. 222:581-597 (1991).

Employing the hybridoma method, a mouse was immunized with the C-terminal 250-amino-acid polypeptide of C. difficile toxin B to elicit lymphocytes that produce or are capable of producing antibodies that will specifically bind to the immunogen (i.e., the C-terminal 250-amino acid polypeptide of toxin B). Other mammals may also be used in generating mAbs according to the disclosure, such as a hamster or macaque monkey. Alternatively, lymphocytes may be immunized in vitro.

Following immunization, lymphocytes were fused with myeloma cells using polyethylene glycol as a fusing agent to form a hybridoma cell (see Goding, Monoclonal Antibodies: Principles and Practice, pp. 59-103 (Academic Press, 1986)). The hybridoma cells thus prepared were seeded and grown in hypoxanthine, aminopterin, and thymidine (HAT medium) culture medium that selected against unfused HGPRT-deficient myeloma cells.

Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M.C.-11 mouse tumors available from the Salk Institute Cell Distribution Center, San Diego, Calif. USA, and SP2/0 or X63-Ag8-653 cells available from the American Type Culture Collection, Rockville, Md. USA.

Culture medium in which hybridoma cells were growing was assayed for production of monoclonal antibodies directed against the C-terminal 250-amino-acid polypeptide of toxin B. Preferably, the binding specificity of monoclonal antibodies produced by hybridoma cells is determined by immunoprecipitation or by an in vitro binding assay, such as a radioimmunoassay (RIA) or enzyme-linked immunosorbent assay (ELISA). The binding affinity of the monoclonal antibody can, for example, be determined by Scatchard analysis (Munson et al., Anal. Biochem., 107:220 (1980)).

After hybridoma cells that produce antibodies of the desired specificity, affinity, and/or activity, were identified, the identified clones were subcloned by limiting dilution procedures and grown by standard methods (Goding, Monoclonal Antibodies: Principles and Practice, pp. 59-103 (Academic Press, 1986)). Culture media for this purpose include, for example, DMEM or RPMI-1640 medium. In addition, the hybridoma cells may be grown in vivo as ascites tumors in an animal. The monoclonal antibodies secreted by the subclones are separated from the culture medium, ascites fluid, or serum by conventional immunoglobulin purification procedures such as, for example, protein G-Sepharose, hydroxylapatite chromatography, gel electrophoresis, dialysis, or affinity chromatography.

It is further contemplated that antibodies of the invention may be used or smaller antigen binding fragments of the antibody, which are well-known in the art and described herein, may be used in the methods according to the disclosure.

### Example 5

### Western Blot Analysis

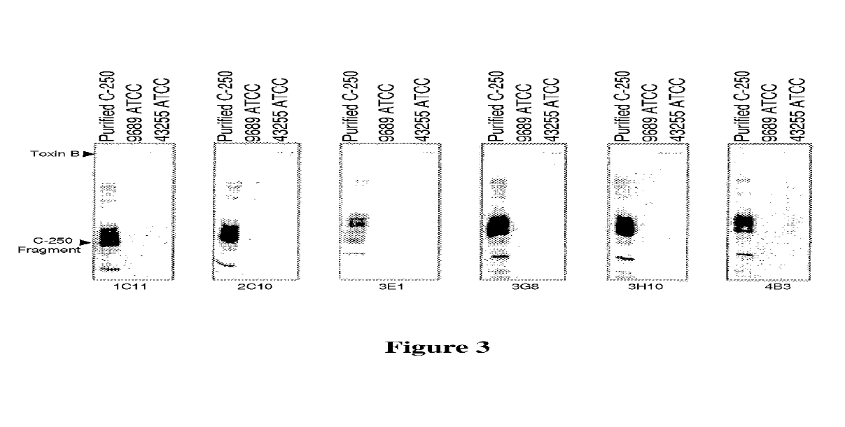

The specificity of the binding of the monoclonal anti-CDB-C250 antibody was assessed by Western blot, using conventional techniques. Separate blots were prepared for the monoclonal anti-CDB-C250 antibodies secreted into the culture supernatant by each of the hybridomas generated in Example 4, i.e., 1C11, 2C10, 3E1, 3G8, 3H10 and 4B3. Each blot contained purified CDB-C250, a crude lysate of a strain of C. difficile (ATCC 9689) that expresses toxin late in the growth cycle at a lower level (Rolfe, et al., Infection and Immunity, 25:191-201, 1979), and a crude lysate of a pathogenic strain of C. difficile (ATCC 43255) that hyper-produces toxins A and B early in the growth phase (Murray et al., BMC Infectious Diseases 9:103 doi:10.1186/1471-2334-9-103, 2009). As shown in FIG. 3, each of the anti-CDB-C250 mAbs specifically bound to purified CDB-C250, although the polypeptide ran as a smear in the SDS-PAGE used to fractionate proteins. Without wishing to be bound by theory, one logical explanation for the smear is that CDB-C250 has a relatively high negative charge at neutral pH and weak binding to SDS. Also of note, the anti-CDB-C250 mAbs bound to intact toxin B in the hyper-producing strain, as expected, but not to intact toxin A, and this binding pattern was the same in both strains. There was no signal for toxin B in the ATCC 9689 strain, but this was not surprising based on the fact that the antibody preparations were crude antibody lysates and the ATCC 9689 strain is not a robust producer of toxin B in vitro. The results demonstrate that the monoclonal antibodies raised by immunization with the C-250 polypeptide fragment of C. difficile toxin B recognized intact toxin B. In addition, the epitope structures recognized are stable after SDS-PAGE and Western blotting.

### Example 6

### In Vitro Cytotoxicity Testing

Early evaluations of CDB-C250 protein expressed in E. coli used protein that was effectively purified to homogeneity. From stocks of lyophilized product, the protein was diluted in physiological buffer and applied in cell culture studies. Using three strains of C. difficile and exposing CDB-C250 to undefined titers of toxin B supernatant obtained from C. difficile cultured in anaerobic chopped meat broth demonstrated that one strain was unaffected in its in vitro action in cell culture, one strain's toxin was partially affected by CDB-C250, and one strain demonstrated definitive inhibition of toxin activity. Thus, the data relating to CDB-C250 establish the potential of CDB-C250 to directly block the cytotoxic effect of C. difficile toxin B.

One of the assays for production of C. difficile toxin B in its various forms, and in particular CDB-C250, is an in vitro assay for cytotoxicity. C. difficile strains are grown to purity, then 3 to 5 colonies are selected and inoculated into anaerobic broth and incubated at 35-37° C. for 3 to 7 days. Cytotoxin testing is performed with the TechLab C. DIFFICILE TOX-B (Toxin/Antitoxin) Kit (TechLab, Blacksburg, Va.). The C. DIFFICILE TOX-B TEST relies on a tissue culture format to detect cytotoxic activity, in the form of cell rounding, in fecal specimens. The test identifies C. difficile toxin B by using specific anti-toxin. Testing on isolated C. difficile colonies is performed using 2-3 mls of anaerobic chopped-meat glucose broth suspension grown with C. difficile and then centrifuged at 4,000×g for 10 minutes and subsequently filtered through a 0.45 μm Spin-X filter. To determine the presence of toxin, two tubes of MRC-5 cells (ViroMed Laboratories) are set up for each sample. Sample alone and sample plus anti-toxin are tested with the TechLab C. difficile Tox-B (Toxin/Antitoxin) Kit. Test results are determined after 24 hours and 48 hours of incubation, according to the manufacturer's instructions. The sample is considered toxigenic if a cytopathic effect (CPE) is observed in the toxin tube and not in the tube containing added anti-toxin. The in vitro cytotoxicity assay is amenable to the assessment of toxin B production by C. difficile isolates, such as C. difficile isolates from patient stool samples. In addition, an ELISA or a modification of the in vitro cytotoxicity assay are useful in assessing the cytotoxiciy of the various recombinantly produced toxin B proteins, peptides or peptide fragments, e.g., CDB-C250. In testing toxin B proteins, routine optimization will reveal the quantity of protein to use in an ELISA or to add to the TOX-B kit reagents to obtain reliable assay results, and the assay can be performed without the need for cell culturing.

An in vitro assay is also available to optimize dosages of the toxin B peptide fragments (CDB-C250, toxin B C-terminal repeat-containing peptides) and of the specific anti-toxin B antibodies. To optimize the dosage of a toxin B fragment, for example, subspecies typing of 100 strains of C. difficile collected from unique patients will be performed using REA and PFGE to define the strain genotypes. The strategy is to select 20 unique strain types representative of those most common in current US circulation and measure their capacity for toxin production after 5 days incubation in anaerobic chopped-meat glucose broth. Five days is chosen so that toxin production is complete and thus permits reproducibility of the experiments over time. The toxin titer chosen for use in this portion of the analysis will be such that each strain's diluted toxin demonstrates 50% destruction of the tissue cells at 48 hours when diluted 1:100 with growth medium. A toxin B fragment such as CDB-C250 protein will then be tested at serially defined concentrations so that the action of toxin B is blocked in at least 80% of the 20 tested C. difficile strains. One of skill in the art will recognize that there are alternative approaches to dosage determination and optimization known in the art, and each of these approaches is contemplated as suitable for use with the diagnostic, prophylactic and therapeutic compounds disclosed herein.

### Example 7

### Testing C. difficile in a Mouse Model

Another measure of the production of C. difficile toxin B in it many forms, e.g., CDB-C250, uses a mouse model. This model was chosen as one of two animal models for use because it relatively closely resembles the full spectrum of human disease in that acute diarrhea as well as chronic diarrhea are represented, and it presents the opportunity for investigating new drug therapy (Steidler et al., Science 289(5483):1352-1355 (2000)). This mouse model is used to assess the prevention of CDI as well as the treatment of CDI using toxin B peptides and fragments, such as CDB-C250 or any of the peptides containing at least one of the repeat motifs found in CDB-C250. As a consequence, animals are tested by administering the polypeptide at the inception of experimental CDI as well as one day into the onset of disease. Following the method of Chen and colleagues (Chen et al., Gastroenterol. 135:1984-1992 (2008)), 9-week-old C57BL/6 female mice are each treated with an antibiotic mixture consisting of kanamycin (0.4 mg/mL), gentamicin (0.035 mg/mL), colistin (850 U/mL), metronidazole (0.215 mg/mL), and vancomycin (0.045 mg/mL) in drinking water for 3 days before clindamycin and C difficile challenge. Clindamycin is administered after a single day of regular water for drinking as a single dose (10 mg/kg) intraperitoneally 1 day before C difficile challenge. Animals are infected by gavage with strains of C difficile and monitored for signs of disease such as diarrhea, hunched posture, wet tail, and weight loss for 10-14 days.

Histopathologic study is done on approximately 50% of the study animals to obtain a valid observation as to the consistent nature of the represented disease. Histologic examination of colonic tissues in mice exposed to C. difficile is expected to demonstrate proliferative ulcerative enteritis with superficial epithelial necrosis and release of inflammatory exudates and necrotic cellular material into the intestinal lumen, as known in the art. Additional indications of CDI are extensive submucosal edema without submucosal inflammation and patchy epithelial necrosis, mucosal proliferation, with the presence of inflammatory cells, as is described for human C. difficile-associated colitis.

### Example 8

### Testing C. difficile in a Hamster Model

The second animal model used to measure C. difficile toxin B production in its several forms, including but not limited to CDB-C250 or any of the peptides containing at least one repeat motif from the CDB-C250 region of toxin B (see FIG. 2), is that of the well-described Syrian Hamster model (Steidler et al., Science 289(5483):1352-1355 (2000), Bermudez-Humaran, Hum. Vacc. 5:264-267 (2009), van Asseldonk et al., Gene 95:155-160 (1990)), following the method described by Razaq and colleagues (van Asseldonk et al.). The rationale for using this model as the second model is that it has become the standard for testing susceptibility to acute CDI disease after antibiotic administration, and the model is recognized by the FDA. This model is useful to assess the ability of CDB-C250 to protect against CDI. Therefore, the polypeptide is only administered at (or before) the inception of CDI and results are compared to controls. C. difficile is inoculated anaerobically onto pre-reduced blood agar plates and incubated at 37° C. until colonies are confluent. The plates are maintained for 3 days to maximize sporulation. The organisms are then harvested, placed into 10 mL of phosphate-buffered saline (PBS) without added calcium or magnesium, washed in PBS, and heat-shocked at 56° C. for 10 minutes to kill surviving vegetative cells. The spores are centrifuged and resuspended in Dulbecco's Modified Eagle Medium (DMEM), aliquoted, and frozen at −80° C. The frozen spores are quantitated before use by plating 100 μL of 10-fold serial dilutions of the spores onto taurocholate fructose agar plates. Spores are diluted in DMEM for orogastric inoculation into hamsters. One μL of food coloring is added to the inoculum for ease of visibility to ensure that hamsters receive the entire dose. For each isolate, hamsters are given 1 dose of clindamycin orogastrically (30 mg/kg) on day 0, to establish susceptibility to CDI. This is followed on day 5 by gastric inoculation with 100 colony-forming units of the designated C. difficile spores. Immediately preceding treatment with clindamycin, bedding is changed, and fecal pellets are collected for culture on C. difficile selective medium. This is done to confirm that hamsters were not colonized with C. difficile before the administration of clindamycin. Hamsters are monitored for signs of C. difficile infection that include stiffness, lying prone, wet tail, diarrhea, and death. Hamsters found lying prone or unresponsive are euthanized.

Histopathologic study is performed on approximately 50% of the study to obtain a valid observation as to the consistent nature of the represented disease. As with the mice, we will assess the comparison of CDB-C250 polypeptide-treated animals (with no prior antimicrobial or C. difficile exposure) to controls so as to demonstrate no adverse effect of the therapy on the colonic mucosa.

### Example 9

### Further Testing of Antibodies Specific to Toxin B

Additional work was carried out on monoclonal antibodies raised against CDB-C250 to determine their reactivities to toxin B from various strains of toxigenic C. difficile. After preliminary evaluation of the six monoclonal antibodies identified in Example 5, monoclonal antibody 3H10 was identified as demonstrating good immunoreactivity against toxin B and the best neutralization of toxin B cytotoxicity. As a result, 3H10 from ascites fluid (2.3 mg of purified mAb from 1 mL fluid) was affinity purified on a toxin B column. The original hybridoma supernatant concentration was about 23 μg/mL. The purified 3H10 antibody was tested for immunoreactivity and found to have a positive reaction to native toxin B at antibody dilutions of 10⁻⁶ to 10⁻⁷. These data correspond to an antibody concentration of about 1 ng/mL and a binding affinity of 1.8×10¹¹ M⁻¹. The mAb was also tested for specificity using an ELISA assay. The 3H10 mAb did not react with purified native toxin A. By immunoblotting, 3H10 was found to react also with denatured toxin B.

Additional experiments were performed to test the capacity of 3H10 to neutralize toxin B in tissue-culture cell-rounding assays. Undiluted (500 μg/mL per assay well) purified antibody showed complete neutralization of cell rounding caused by a 10⁻⁷ dilution of toxin B and partial neutralization of rounding caused by a 10⁻⁶ dilution of toxin B.

A biotinylated 3H10 mAb was also conjugated to plates coated with Streptavidin to yield Streptavidin:biotin-3H10. Both 0.5 and 1 μg/well 3H10-biotin showed equivalent ability to capture toxin B as a toxin A/B II polyclonal antibody mix developed at TECHLAB.

These new data add convincing evidence that the CDB-C250 peptide represents a highly specific domain structure of toxin B with an important role in cytotoxicity. This confirmation further justifies the expectation that CDB-C250 peptide, and peptides comprising at least one repeat motif from the CDB-C250 domain of toxin B, will provide effective prophylaxis and/or treatment of CDI in subjects, including human patients and non-human animals.

### Example 10

### C. difficile Strains

Each archived C. difficile strain is plated to a pre-reduced cycloserine-cefoxitin-fructose agar (CCFA-VA formulation) and anaerobic blood agar media. Plates are then incubated anaerobically at 35-37° C. for up to 72 hours to assure purity of the archived strains. Colonies are confirmed by Gram stain, aerotolerance, and a Pro-disk test (Key Scientific). Two methods are available to ensure that the growth and handling of various C. difficile species does not lead to confusion and to ensure that there is no uncertainty in the classification of the C. difficile genotypes. Restriction Endonuclease Analysis (REA) typing is one standardized method that is performed, e.g., with the HindIII restriction enzyme, as would be known in the art. Briefly, brain heart infusion broth is inoculated with 3-5 colonies from an anaerobic blood agar plated and then incubated overnight. Cells are washed in TE (10 mM Tris-HCl, 1 mM EDTA [pH 8.0]), re-suspended in 0.1 mL of TE with lysozyme (50 mg/mL; Sigma-Aldrich), incubated for 30 min at 35° C., mixed with 0.5 mL of GES solution (guanidine thiocyanate, 0.6 g/mL; EDTA, 100 mM; sarcosyl, 0.5%, vol/vol), incubated for 10 minutes at room temperature, mixed with 0.75 mL of ammonium acetate (7.5 M), and held on ice for 10 minutes. DNA is extracted with phenol:chloroform:isoamyl alcohol (25:24:1) and precipitated with cold 2-propanol. For restriction digestion, DNA (10 to 20 RII) is incubated with HindIII (Bethesda Research Laboratories, Gaithersburg, Md.) according to the manufacturer's recommendations, except that 20 U of enzyme is used and 3 RI of spermidine (100,μg/mL; Sigma) is added. The resulting restriction fragments are resolved in a 0.7% agarose gel and the gel is then stained with ethidium bromide and photographed under UV light, producing a characteristic banding pattern for each isolate that is visually compared with the patterns of previously identified REA types. Isolates are categorized into ‘groups’ (letter designation) if the patterns had <6 band differences (similarity index >90%) and into specific ‘types’ (number designation following the ‘group’ letter) based on unique, identical REA patterns.

Pulsed-Field Gel Electrophoresis (PFGE) is another standard method for C. difficile genotyping and will be accomplished following standard methods. Briefly, isolates are inoculated into pre-reduced brain heart infusion broth and incubated at 37° C. The optical density is monitored in a spectrophotometer. When growth reaches mid-exponential phase (optical density at 540 nm 2 0.500), about 7 hours after inoculation, the organisms are collected by centrifugation at 4° C. and then processed for DNA using conventional methods. C. difficile DNA in agarose is digested with SmaI (New England Biolabs, Cambridge, Mass.), and the resulting macrorestriction fragments are resolved by PFGE. The gels are electrophoresed for 22 hours in a contour-clamped homogeneous electric field apparatus (CHEF DRII; Bio-Rad, Richmond, Calif.) at 6.0 V/cm, with initial and final switch times of 20 and 70 s, respectively, and linear ramping. The gels are stained with ethidium bromide and photographed under UV light. SmaI-digested S. aureus (ATCC 8325) is used as a molecular weight size standard.

Numerous modifications and variations of the disclosure are possible in view of the above teachings and are within the scope of the claims. The above-described embodiments are not intended to limit the claims in any way. The entire disclosure of all publications cited herein are hereby incorporated by reference.

1. An antibody or antibody fragment that specifically binds to the C-terminal 250-amino-acid region of Clostridium difficile toxin B and that does not detectably bind to C. difficile toxin A.

2. The antibody or antibody fragment according to claim 1 wherein the antibody or antibody fragment is a monoclonal antibody or antibody fragment.

3. The antibody or antibody fragment according to claim 1 wherein the antibody or antibody fragment specifically binds to a polypeptide comprising the sequence selected from the group consisting of SEQ ID NOS:3-13.

4. The antibody or antibody fragment according to claim 2 wherein the antibody or antibody fragment is produced by a hybridoma selected from the group consisting of the 3H10 hybridoma, the 1C11 hybridoma, the 2C10 hybridoma, the 3E1 hybridoma, the 3G8 hybridoma and the 4B3 hybridoma.

5. An antibody or antibody fragment that specifically binds to an epitope to which the antibody or antibody fragment according to claim 1 specifically binds.

6. The antibody or antibody fragment according to claim 1, further comprising a second polypeptide covalently bound to the antibody or antibody fragment in a fusion polypeptide, wherein the second polypeptide is a cytotoxic polypeptide.

7. The antibody or antibody fragment of claim 1 that binds the C-terminal 250-amino-acid region of Clostridium difficile toxin B with an affinity of at least 10⁸ M⁻¹ that comprises:
(a) a heavy chain CDR1 amino acid sequence selected from the group consisting of SEQ ID NOS: 39, 42, 45, 48, 51 and a variant thereof in which at most two amino acids have been changed, or a consensus sequence thereof;
(b) a heavy chain CDR2 amino acid sequence selected from the group consisting of SEQ ID NOS: 40, 43, 46, 49, 52 and a variant thereof in which at most two amino acids have been changed or a consensus sequence thereof; and
(c) a heavy chain CDR3 amino acid sequence selected from the group consisting of SEQ ID NOS: 41, 44, 47, 50, 53 and a variant thereof in which at most two amino acids have been changed, or a consensus sequence thereof.

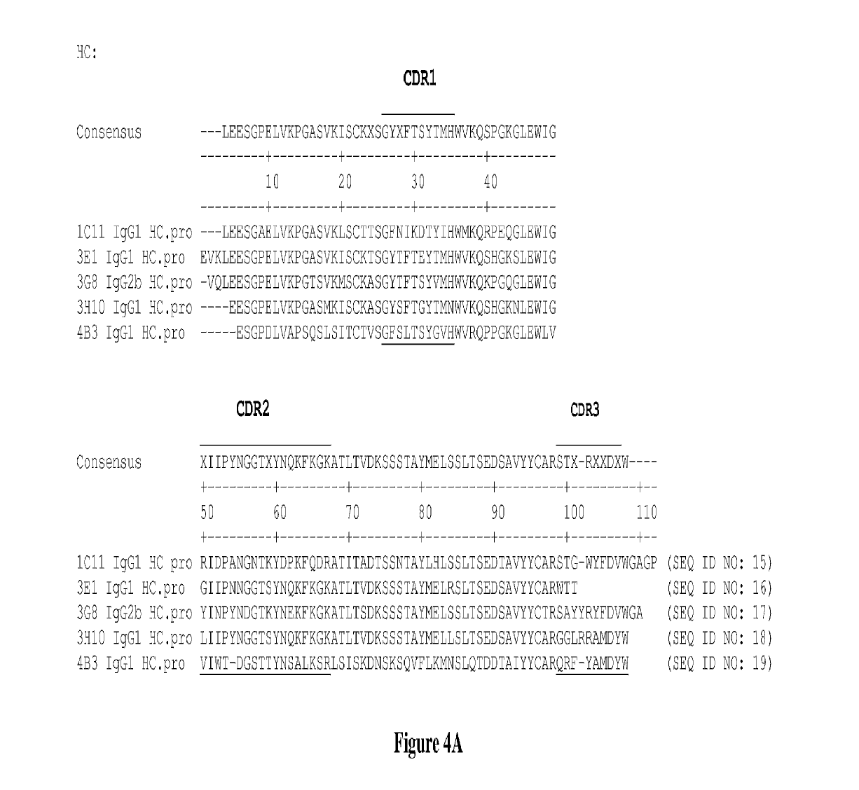

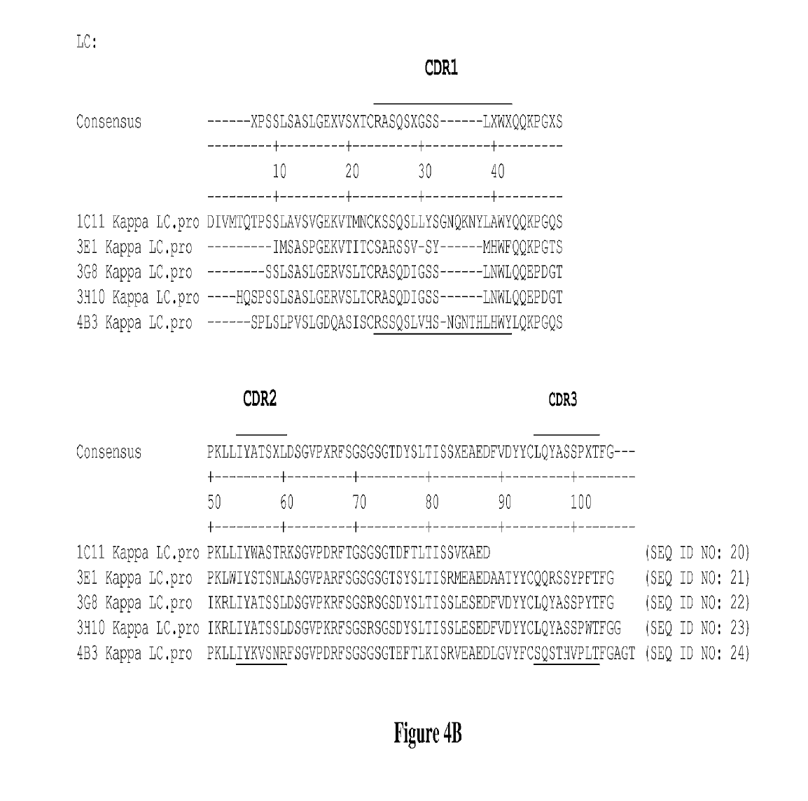

8. The antibody or antibody fragment of claim 7 wherein one or more of said heavy chain CDR1, CDR2 or CDR3 amino acid sequences is a consensus sequence set forth in FIG. 4.

9. The antibody or antibody fragment of claim 7 wherein
(a) an amino acid in a heavy chain CDR1 amino acid sequence is replaced with an amino acid from a corresponding position within a different heavy chain CDR1 amino acid sequence set forth in FIG. 4;
(b) an amino acid in a heavy chain CDR2 amino acid sequence is replaced with an amino acid from a corresponding position within a different heavy chain CDR2 amino acid sequence set forth in FIG. 4; or
(c) an amino acid in a heavy chain CDR3 amino acid sequence is replaced with an amino acid from a corresponding position within a different heavy chain CDR3 amino acid sequence set forth in FIG. 4.

10. The antibody or antibody fragment of claim 7 that comprises an amino acid sequence at least 95% identical to a heavy chain variable region amino acid sequence set forth in FIG. 4.

11. The antibody or antibody fragment of claim 10 that comprises a heavy chain variable region amino acid sequence set forth in FIG. 4.

12. The antibody or antibody fragment of claim 7 in which one or more heavy chain framework amino acids have been replaced with corresponding amino acid(s) from another human antibody heavy chain framework amino acid sequence.

13. The antibody or antibody fragment of claim 1 that binds the C-terminal 250-amino-acid region of Clostridium difficile toxin B with an affinity of at least 10⁸ M⁻¹ that comprises:
(a) a light chain CDR1 amino acid sequence selected from the group consisting of SEQ ID NOS: 25, 27, 30, 33, 36 and a variant thereof in which at most two amino acids have been changed;
(b) a light chain CDR2 amino acid sequence selected from the group consisting of SEQ ID NOS: 26, 28, 31, 34, 37 and a variant thereof in which at most two amino acids have been changed; and
(c) a light chain CDR3 amino acid sequence selected from the group consisting of SEQ ID NOS: 29, 32, 35, 38 and a variant thereof in which at most two amino acids have been changed.

14. The antibody or antibody fragment of claim 13 wherein one or more of said light chain CDR1, CDR2 or CDR3 amino acid sequences is a consensus sequence set forth in FIG. 4.

15. The antibody or antibody fragment of claim 13 wherein
(a) an amino acid in a light chain CDR1 amino acid sequence is replaced with an amino acid from a corresponding position within a different light chain CDR1 amino acid sequence set forth in FIG. 4;
(b) an amino acid in a light chain CDR2 amino acid sequence is replaced with an amino acid from a corresponding position within a different light chain CDR2 amino acid sequence set forth in FIG. 4; or
(c) an amino acid in a light chain CDR3 amino acid sequence is replaced with an amino acid from a corresponding position within a different light chain CDR3 amino acid sequence set forth in FIG. 4.

16. The antibody or antibody fragment of claim 13 that comprises an amino acid sequence at least 95% identical to a light chain variable region amino acid sequence set forth in FIG. 4.

17. The antibody or antibody fragment of claim 13 that comprises a light chain variable region amino acid sequence set forth in FIG. 4.

18. The antibody or antibody fragment of claim 13 in which one or more light chain framework amino acids have been replaced with corresponding amino acid(s) from another human antibody light chain framework amino acid sequence.

19. A hybridoma producing the antibody or antibody fragment according to any claim 1.

20. The hybridoma according to claim 19 wherein the hybridoma is selected from the group consisting of the 3H10 hybridoma, the 1C11 hybridoma, the 2C10 hybridoma, the 3E1 hybridoma, the 3G8 hybridoma and the 4B3 hybridoma.

21. A polypeptide comprising a fragment of Clostridium difficile toxin B, wherein the fragment consists of a sequence selected from the group consisting of SEQ ID NOS:2-13.

22. A polynucleotide comprising the polynucleotide sequence encoding an amino acid sequence selected from the group consisting of SEQ ID NOS:2-13.

23. A vector comprising the polynucleotide according to claim 22.

24. A host cell comprising the vector according to claim 23.

25. A method for detecting the presence of Clostridium difficile toxin B in a sample comprising:
(a) contacting said sample with an antibody or antibody fragment according to claim 1 under conditions suitable for binding; and
(b) detecting the binding of said antibody or antibody fragment to Clostridium difficile toxin B.

26. The method according to claim 25, wherein said sample is a stool sample or a fluid exposed to a stool sample.

27. A method for diagnosing Clostridium difficile infection (CDI) in a subject comprising:
(a) obtaining a biological sample from said subject;
(b) contacting said sample with an antibody or antibody fragment according to claim 1 under conditions suitable for binding; and
(c) detecting the binding of said antibody or antibody fragment to Clostridium difficile toxin B, wherein the binding of said antibody or antibody fragment to toxin B is diagnostic of Clostridium difficile infection.

28. The method according to claim 27 wherein the biological sample is a stool sample or a fluid exposed to a stool sample.

29. A method for diagnosing Clostridium difficile infection (CDI) in a subject comprising:
(a) obtaining a biological sample from said subject;
(b) adding to the sample a pair of PCR primers capable of amplifying a region of Clostridium difficile tcdB between 8-750 nucleotides in length at the 3′ end of the tcdB coding region under polymerase chain reaction (PCR) conditions;
(c) performing a PCR; and
(d) diagnosing CDI if Clostridium difficile is detected in the sample.

30. A method for vaccinating a subject comprising administering an immunologically effective amount of the polypeptide according to claim 21 to a subject.

31. A method of preventing or treating Clostridium difficile Infection (CDI) comprising administering a therapeutically effective amount of the antibody or antibody fragment according to claim 1 to a subject.

32. A method of preventing or treating Clostridium difficile Infection (CDI) comprising administering a therapeutically effective amount of the polypeptide according to claim 21 to a patient.

33-34. (canceled)
